(19) United States
(12) Patent Application Publication
(10) Pub. No.: US 2026/0148852 A1
(43) Pub. Date: May 28, 2026

(54) ORGANIC MOLECULE EXPOSOMICS

(71) Applicant: LINUS BIOTECHNOLOGY INC., North Brunswick, NJ (US)

(72) Inventors: Manish ARORA, New Rochelle, NY (US); Paul CURTIN, Jersey City, NJ (US); Austen CURTIN, Jersey City, NJ (US); Christine AUSTIN, New York, NY (US); Joanna Franklin ELLIS, Brooklyn, NY (US)

(21) Appl. No.: 19/307,658

(22) Filed: Aug. 22, 2025

Related U.S. Application Data
(63) Continuation of application No. PCT/US2024/017327, filed on Feb. 26, 2024.
(60) Provisional application No. 63/487,150, filed on Feb. 27, 2023.

(51) Int. Cl. G16H 50/20; G01N 33/68; G06N 20/00; G16C 20/20; G16C 20/70; G16H 20/10; G16H 50/70

(52) U.S. Cl. CPC G16H 50/20; G01N 33/6815; G06N 20/00; G16C 20/20; G16C 20/70; G16H 20/10; G16H 50/70

(57) ABSTRACT

Presented and described herein are methods of detecting, collecting, and analyzing organic molecule signature exposomics of a subject.

### CROSS REFERENCE

This application is a continuation of PCT International Application No. PCT/US2024/017327, filed Feb. 26, 2024, which application claims the benefit of U.S. Provisional Patent Application No. 63/487,150, filed Feb. 27, 2023, each of which are incorporated by reference herein in their entirety.

### BACKGROUND

Organic molecules have an important role in many biological processes having structural and functional significance for human metabolic, physiologic, and/or molecular pathways. Changes in the concentration of organic molecules of a subject's may be influenced by diet, taking pharmaceutical compounds, environmental exposure, and/or the development or presence of disease.

Accordingly, there exists an unmet need of systems and devices capable of rapidly and non-invasively assessing the rich dataset of a subject's organic molecule signatures to classify one or more molecular and/or biological phenotypes of a subject.

### SUMMARY

Aspects of the disclosure provided herein comprise a method for classifying a phenotype of a subject, comprising: obtaining or collecting one or more organic molecule signatures from a plurality of positions along a biological sample of a subject; and determining the phenotype of the subject from the one or more organic molecule signatures of the subject. In some embodiments, the one or more organic molecule signatures comprise a molecular signature of a molecule with a mass to charge ratio of at least 50 Daltons (Da). In some embodiments, the one or more organic molecule signatures comprise one or more time-resolved organic molecule signatures. In some embodiments, a light source is used to obtain or collect the one or more organic molecule signatures from the plurality of positions along the biological sample. In some embodiments, the light source comprises a laser, pulsed laser, continuous wave laser, or any combination thereof. In some embodiments, the phenotype of the subject comprises molecular phenotype, physiologic phenotype, a behavioral phenotype, a disease phenotype, a healthy phenotype, an exposure phenotype, one or more upregulated or downregulated physiological pathways, pharmaceutical response, nutraceutical response, presence of exogenous compounds, presence of endogenous compounds, presence of inflammation, or any combination thereof. In some embodiments, the disease phenotype comprises autism spectrum disorder (ASD), attention-deficit/hyperactivity disorder, amyotrophic lateral sclerosis, or any combination thereof. In some embodiments, the exogenous compounds comprise nicotine, melamine, or any combination thereof. In some embodiments, the endogenous compounds comprise endogenous metabolites, signaling molecules, or any combination thereof. In some embodiments, the signaling molecules comprise hypotaurine. In some embodiments, the endogenous metabolites comprise creatinine. In some embodiments, the nutraceutical comprises agmatine. In some embodiments, the pharmaceutical comprises a pharmaceutical to treat heart burn, acid reflux, peptic ulcers, or any combination thereof. In some embodiments, the pharmaceutical comprises Betazole. In some embodiments, the exposure phenotype comprises exposure of the subject to environmental chemicals. In some embodiments, the biological sample comprises hair, teeth, fingernails, toenails, or any combination thereof samples. In some embodiments, obtaining or collecting comprises conducting matrix assisted laser desorption ionization-time of flight mass spectrometry (MALDI-ToF MS), laser ablation electrospray ionization (LAESI), protein fluorescence assays, or any combination thereof with the biological sample. In some embodiments, the subject has been administered or is taking a pharmaceutical, nutraceutical, or a combination thereof to treat a condition of the subject, and wherein the phenotype of the subject comprises a response to the pharmaceutical, nutraceutical, or a combination thereof. In some embodiments, the phenotype is used to determine an adjustment of the pharmaceutical, nutraceutical, or a combination thereof, to ameliorate the condition of the subject. In some embodiments, the adjustment of the pharmaceutical, nutraceutical, or a combination thereof comprises administering modified or new pharmaceutical, nutraceutical, or a combination thereof. In some embodiments, the one or more organic molecule signatures comprise temporal concentrations of one or more organic molecules obtained or collected from the biological sample of the subject. In some embodiments, determining the phenotype of the subject comprises: training a predictive model with one or more organic molecule signatures and associated phenotype labels of a set of subjects different than the subject; and providing the subject's one or more organic molecule signatures to the trained predictive model and outputting the phenotype of the subject.

Aspects of the disclosure provided herein comprise a method for classifying a phenotype of a subject, comprising: obtaining or collecting one or more molecular signatures of molecules with a mass to charge ratio of at least 50 Da from a biological sample of a subject when one or more probes are disposed on the biological sample; and determining the phenotype of the subject from the one or more molecular signatures of the subject. In some embodiments, the one or more molecular signatures comprise one or more organic molecule signatures. In some embodiments, a light source is used to obtain or collect the one or more molecular signatures from the one or more probes disposed on the biological sample. In some embodiments, the light source comprises a laser, pulsed laser, continuous wave laser, or any combination thereof. In some embodiments, the phenotype of the subject comprises a molecular phenotype, physiologic phenotype, behavioral phenotype, a disease phenotype, a healthy phenotype, an exposure phenotype, one or more upregulated or downregulated physiological pathways, pharmaceutical response, nutraceutical response, presence of exogenous compounds, presence of endogenous compounds, presence of inflammation, or any combination thereof. In some embodiments, the disease phenotype comprises autism spectrum disorder (ASD), attention-deficit/hyperactivity disorder, amyotrophic lateral sclerosis, or any combination thereof. In some embodiments, the exogenous compounds comprise nicotine, melamine, or any combination thereof. In some embodiments, the endogenous compounds comprise endogenous metabolites, signaling molecules, or any combination thereof. In some embodiments, the signaling molecules comprise hypotaurine. In some embodiments, the endogenous metabolites comprise creatinine. In some embodiments, the nutraceutical comprises agmatine. In some embodiments, the pharmaceutical comprises a pharmaceutical to treat heart burnt, acid reflux, peptic ulcers, or any combination thereof. In some embodiments, the pharmaceutical comprises Betazole. In some embodiments, betazole exposure phenotype comprises exposure of the subject to environmental chemicals. In some embodiments, the biological sample comprises hair, teeth, fingernails, toenails, or any combination thereof samples. In some embodiments, obtaining or collecting comprise conducting a protein fluorescence assay with the one or more probes. In some embodiments, the subject has been administered or is taking a pharmaceutical, nutraceutical, or a combination thereof to treat a condition of the subject, and wherein the phenotype of the subject comprises a response to the pharmaceutical, nutraceutical, or a combination thereof. In some embodiments, the phenotype is used to determine an adjustment of the pharmaceutical, nutraceutical, or a combination thereof, to ameliorate the condition of the subject. In some embodiments, the adjustment of the pharmaceutical, nutraceutical, or a combination thereof comprises administering modified or new pharmaceutical, nutraceutical, or a combination thereof. In some embodiments, determining the phenotype of the subject comprises: training a predictive model with one or more molecular signatures and associated phenotype labels of a set of subjects different than the subject; and providing the subject's one or more molecular signatures to the trained predictive model and outputting the phenotype of the subject.

Aspects of the disclosure provided herein comprise a system for classifying a phenotype of a subject, comprising: one or more processor and memory storing one or more programs for execution by the one or more processors, the one or more programs comprising instructions to: (i) obtain or collect one or more organic molecule signatures of a biological sample of the subject from a plurality of positions along the biological sample; and (ii) determine the phenotype of the subject from the one or more organic molecule signatures of the subject. In some embodiments, the one or more organic molecule signatures comprise a molecular signature of a molecule with mass to charge ratio of at least 50 Daltons (Da). In some embodiments, the one or more organic molecule signatures comprise one or more time-resolved organic molecule signatures. In some embodiments, a light source is used to obtain or collect the one or more organic molecule signatures from the plurality of positions along the biological sample. In some embodiments, the light source comprises a laser, pulsed laser, continuous wave laser, or any combination thereof. In some embodiments, the phenotype of the subject comprises a molecular phenotype, physiologic phenotype, behavioral phenotype, a disease phenotype, a healthy phenotype, an exposure phenotype, one or more upregulated or downregulated physiological pathways, pharmaceutical response, nutraceutical response, presence of exogenous compounds, presence of endogenous compounds, presence of inflammation, or any combination thereof. In some embodiments, the disease phenotype comprises autism spectrum disorder (ASD), attention-deficit/hyperactivity disorder, amyotrophic lateral sclerosis, or any combination thereof. In some embodiments, the exogenous compounds comprise nicotine, melamine, or any combination thereof. In some embodiments, the endogenous compounds comprise endogenous metabolites, signaling molecules, or any combination thereof. In some embodiments, the signaling molecules comprise hypotaurine. In some embodiments, the endogenous metabolites comprise creatinine. In some embodiments, the nutraceutical comprises agmatine. In some embodiments, the pharmaceutical comprises a pharmaceutical to treat heart burnt, acid reflux, peptic ulcers, or any combination thereof. In some embodiments, the pharmaceutical comprises Betazole. In some embodiments, the exposure phenotype comprises exposure of the subject to environmental chemicals. In some embodiments, the biological sample comprises hair, teeth, fingernails, toenails, or any combination thereof samples. In some embodiments, obtaining or collecting comprises conducting matrix assisted laser desorption ionization-time of flight mass spectrometry (MALDI-ToF MS), laser ablation electrospray ionization (LAESI), protein fluorescence assays, or any combination thereof with the biological sample. In some embodiments, the subject has been administered or is taking a pharmaceutical, nutraceutical, or a combination thereof to treat a condition of the subject, and wherein the phenotype of the subject comprises a response to the pharmaceutical, nutraceutical, or a combination thereof. In some embodiments, the phenotype is used to determine an adjustment of the pharmaceutical, nutraceutical, or a combination thereof, to ameliorate the condition of the subject. In some embodiments, the adjustment of the pharmaceutical, nutraceutical, or a combination thereof comprises administering modified or new pharmaceutical, nutraceutical, or a combination thereof. In some embodiments, the one or more organic molecule signatures comprise temporal concentrations of one or more organic molecules obtained or collected from the biological sample of the subject. In some embodiments, determining the phenotype of the subject comprises: training a predictive model with one or more organic molecule signatures and associated phenotype labels of a set of subjects different than the subject; and providing the subject's one or more organic molecule signatures to the trained predictive model and outputting the phenotype of the subject.

Aspects of the disclosure provided herein comprise a system for classifying a phenotype of a subject, comprising: one or more processor and memory storing one or more programs for execution by the one or more processors, the one or more programs comprising instructions to: (i) obtain or collect one or more molecular signatures of molecules with a mass to charge ratio of at least 50 Da from a biological sample of a subject when one or more probes are disposed on the biological sample; and (ii) determine the phenotype of the subject from the one or more molecular signatures of the subject. In some embodiments, the one or more molecular signatures comprise one or more organic molecule signatures. In some embodiments, a light source is used to obtain or collect the one or more molecular signatures from the biological sample. In some embodiments, the light source comprises a laser, pulsed laser, continuous wave laser, or any combination thereof. In some embodiments, the phenotype of the subject comprises a molecular phenotype, physiologic phenotype, behavioral phenotype, a disease phenotype, a healthy phenotype, an exposure phenotype, one or more upregulated or downregulated physiological pathways, pharmaceutical response, nutraceutical response, presence of exogenous compounds, presence of endogenous compounds, presence of inflammation, or any combination thereof. In some embodiments, the disease phenotype comprises autism spectrum disorder (ASD), attention-deficit/hyperactivity disorder, amyotrophic lateral sclerosis, or any combination thereof. In some embodiments, the exogenous compounds comprise nicotine, melamine, or any combination thereof. In some embodiments, the endogenous compounds comprise endogenous metabolites, signaling molecules, or any combination thereof. In some embodiments, the signaling molecules comprise hypotaurine. In some embodiments, the endogenous metabolites comprise creatinine. In some embodiments, the nutraceutical comprises agmatine. In some embodiments, the pharmaceutical comprises a pharmaceutical to treat heart burnt, acid reflux, peptic ulcers, or any combination thereof. In some embodiments, the pharmaceutical comprises Betazole. In some embodiments, the exposure phenotype comprises exposure of the subject to environmental chemicals. In some embodiments, the biological sample comprises hair, teeth, fingernails, toenails, or any combination thereof samples. In some embodiments, the instruction of obtain or collect comprise conducting a protein fluorescence assay with the one or more probes. In some embodiments, the subject has been administered or is taking a pharmaceutical, nutraceutical, or a combination thereof to treat a condition of the subject, and wherein the phenotype of the subject comprises a response to the pharmaceutical, nutraceutical, or a combination thereof. In some embodiments, the phenotype is used to determine an adjustment of the pharmaceutical, nutraceutical, or a combination thereof, to ameliorate the condition of the subject. In some embodiments, the adjustment of the pharmaceutical, nutraceutical, or a combination thereof comprises administering modified or new pharmaceutical, nutraceutical, or a combination thereof. In some embodiments, the instruction of determine the phenotype of the subject comprises: train a predictive model with one or more molecular signatures and associated phenotype labels of a set of subjects different than the subject; and provide the subject's one or more molecular signatures to the trained predictive model and output the phenotype of the subject.

Aspects of the disclosure provided herein comprise a method of training an untrained or partially untrained machine learning algorithm or predictive model, comprising: at a computer system having one or more processors, and memory storing one or more programs for execution by the one or more processors: collecting or obtaining one or more organic molecule signatures of a biological sample of a plurality of training subjects, wherein a first subset of training subjects in the plurality of training subjects have a first phenotype associated with the one or more organic molecule signatures and a second subset of training subjects in the plurality of training subjects have a second phenotype associated with the one or more organic molecule signatures; and training the untrained or partially untrained machine learning algorithm or predictive model with the one or more organic molecule signatures of the plurality of training subjects and the corresponding first phenotype and second phenotype associated with the one or more organic molecule signatures thereby producing or generating a trained predictive model. In some embodiments, the one or more organic molecule signatures comprise one or more organic molecule signatures of molecules with a mass to charge ratio of at least 50 Da. In some embodiments, the one or more organic molecule signatures comprise one or more time-resolved organic molecule signatures. In some embodiments, a light source is used to obtain or collect the one or more organic molecule signatures from the biological sample of the plurality of training subjects. In some embodiments, the light source comprises a laser, pulsed laser, continuous wave laser, or any combination thereof. In some embodiments, the first phenotype or the second phenotype comprise a molecular phenotype, physiologic phenotype, a behavioral phenotype, a disease phenotype, a healthy phenotype, an exposure phenotype, one or more upregulated or downregulated physiological pathways, pharmaceutical response, nutraceutical response, presence of exogenous compounds, presence of endogenous compounds, presence of inflammation, or any combination thereof. In some embodiments, the disease phenotype comprises autism spectrum disorder (ASD), attention-deficit hyperactivity disorder, amyotrophic lateral sclerosis, or any combination thereof. In some embodiments, the exogenous compounds comprise nicotine, melamine, or any combination thereof. In some embodiments, the endogenous compounds comprise endogenous metabolites, signaling molecules, or any combination thereof. In some embodiments, the signaling molecules comprise hypotaurine. In some embodiments, the endogenous metabolites comprise creatinine. In some embodiments, the nutraceutical comprises agmatine. In some embodiments, the pharmaceutical comprises a pharmaceutical to treat heart burn, acid reflux, peptic ulcers, or any combination thereof. In some embodiments, the pharmaceutical comprises Betazole. In some embodiments, the exposure phenotype comprises exposure of the subject to environmental chemicals. In some embodiments, the biological sample comprises hair, teeth, fingernails, toenails, or any combination thereof samples. In some embodiments, obtaining or collecting comprises conducting matrix assisted laser desorption ionization-time of flight mass spectrometry (MALDI-ToF MS), laser ablation electrospray ionization (LAESI), protein fluorescence assays, or any combination thereof with the biological sample. In some embodiments, the plurality of training subjects have been administered or is taking a pharmaceutical, nutraceutical, or a combination thereof to treat a condition of the plurality of training subjects, and wherein the first phenotype or the second phenotype of the plurality of training subjects comprises a response to the pharmaceutical, nutraceutical, or a combination thereof. In some embodiments, the first phenotype or the second phenotype is used to determine an adjustment of the pharmaceutical, nutraceutical, or a combination thereof, to ameliorate the condition of the subject. In some embodiments, the adjustment of the pharmaceutical, nutraceutical, or a combination thereof comprises administering modified or new pharmaceutical, nutraceutical, or a combination thereof. In some embodiments, the one or more organic molecule signatures comprise temporal concentrations of the one or more organic molecules signatures obtained or collected from the biological sample of the plurality of training subjects.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a flow diagram for a method of determining a phenotype of a subject from one or more organic molecule signatures, as described in some embodiments herein.

FIG. 2 shows a flow diagram for a method of determining a phenotype of a subject from one or more molecular signatures, as described in some embodiments herein.

FIGS. 3A-3B show a representative concentration of hypotaurine organic molecule signature (FIG. 3A) collected across a biological sample using the methods, device, and/or systems, described herein, and the comparison of an average hypotaurine concentration compared across control and autism spectrum disorder groups (FIG. 3B), as described in some embodiments herein.

FIGS. 4A-4B show a representative concentration of melamine organic molecule signature (FIG. 4A) collected across a biological sample using the methods, device, and/or systems, described herein, and the comparison of an average melamine concentration compared across control and autism spectrum disorder groups (FIG. 4B), as described in some embodiments herein.

FIGS. 5A-5B show a representative concentration of agmatine organic molecule signature (FIG. 5A) collected across a biological sample using the methods, device, and/or systems, described herein, and the comparison of an average agmatine concentration compared across control and autism spectrum disorder groups (FIG. 5B), as described in some embodiments herein.

FIGS. 6A-6B show a representative concentration of Betazole organic molecule signature (FIG. 6A) collected across a biological sample using the methods, device, and/or systems, described herein, and the comparison of an average Betazole concentration compared across control and autism spectrum disorder groups (FIG. 6B), as described in some embodiments herein.

FIGS. 7A-7B show a representative concentration of creatinine organic molecule signature (FIG. 7A) collected across a biological sample using the methods, device, and/or systems, described herein, and the comparison of an average creatinine concentration compared across control and autism spectrum disorder groups (FIG. 7B), as described in some embodiments herein.

FIGS. 8A-8B show a representative concentration of nicotine organic molecule signature (FIG. 8A) collected across a biological sample using the methods, device, and/or systems, described herein, and the comparison of an average nicotine concentration compared across non-smoker and smoker groups (FIG. 8B), as described in some embodiments herein.

FIG. 9 shows a receiver operating characteristic curve and corresponding area under the curve performance of a predictive model trained with one or more organic molecule signatures when classifying a subject with autism spectrum disorder, as described in some embodiments herein.

FIGS. 10A-10B show composite indexes derived from a plurality of organic molecules between control and subjects with autism spectrum disorder (FIG. 10A) and the associated feature importance for each organic molecule of the plurality of organic molecules considered in the composite index (FIG. 10B), as described in some embodiments herein.

FIGS. 11A-11B show composite indexes derived from a plurality of organic molecules between control and subjects with amyotrophic lateral sclerosis (FIG. 11A) and the associated feature importance for each organic molecule of the plurality of organic molecules considered in the composite index (FIG. 11B), as described in some embodiments herein.

FIG. 12 shows a computer system configured to implement the methods of the disclosure, as described in some embodiments herein.

### DETAILED DESCRIPTION

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease. In some instances, the subject may comprise a set or group of one or more subjects.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The methods and/or systems described herein measure, detect, and/or obtain one or more molecular signatures (e.g., one or more organic molecule signatures) of a biological sample of one or more subjects. In some cases, the one or more molecular signatures may comprise a concentration of one or more molecules (e.g., one or more organic molecules). In some cases, the methods and/or systems described herein may measure, detect, and/or obtain concentrations of one or more molecular signatures non-invasively. The one or more molecular signatures may be used as features to classify and/or predict a phenotype of a subject. In some cases, the phenotype of the subject may comprise a molecular phenotype, physiologic phenotype, a behavior phenotype, a disease phenotype, a health phenotype, an exposure phenotype, one or more upregulated or downregulated physiological pathways, pharmaceutical response, nutraceutical response, presence of exogenous compounds, presence of endogenous compounds, presence of inflammation, or any combination thereof. In some cases, the disease phenotype may comprise autism spectrum disorder (ASD), attention-deficit/hyperactivity disorder, amyotrophic lateral sclerosis (ALS), cancer, or any combination thereof. In some cases, the exogenous compounds may comprise nicotine, melamine, or a combination thereof. In some instances, the endogenous compounds may comprise metabolites, signaling molecules, or any combination thereof. The signaling molecules may comprise hypotaurine. In some cases, the endogenous metabolites may comprise creatinine. In some instances, the nutraceutical may comprise agmatine. In some instances, the pharmaceutical may comprise a pharmaceutical to treat heart burn, acid reflux, peptic ulcers, or any combination thereof. In some cases, the pharmaceutical may comprise Betazole. In some instances, the exposure phenotype may comprise exposure of the subject to environmental chemicals.

In some cases, a subject's phenotype may be used to determine the subject's response to a pharmaceutical and/or nutraceutical administered to the subject. In some cases, a subject's phenotype may be used to inform how to adjust e.g., change the pharmaceutical and/or nutraceutical compound entirely, add to the pharmaceutical and/or nutraceutical compound, identify a new pharmaceutical and/or nutraceutical compound to administer to the subject, change the dosing regimen of the pharmaceutical nutraceutical compound, or any combination thereof. In some instances, the pharmaceutical and/or nutraceutical administered may ameliorate a disease or one or more symptoms of a disease and/or condition of the subject.

In some instances, the biological sample of the subject may comprise a biological tissue, biological tissue biopsy, liquid biopsy, or any combination thereof. In some cases, the biological tissue may comprise fingernails, toenails, hair, teeth, or any combination thereof.

In some cases, a light source may be used to obtain and/or collect the one or more molecular signatures from the biological sample. In some cases, the light source may comprise a laser, pulsed laser, continuous wave laser, or any combination thereof. In some instances, the one or more molecular signatures may be detected, measured, and/or obtained using matrix assisted laser desorption ionization time of flight mass spectrometry (MADLI ToF MS), laser ablation electrospray ionization (LAESI), trapped ion mobility spectrometry (TIMS), time-of-flight (TOF) mass spectrometry, protein fluorescence assays, or any combination thereof.

### Methods

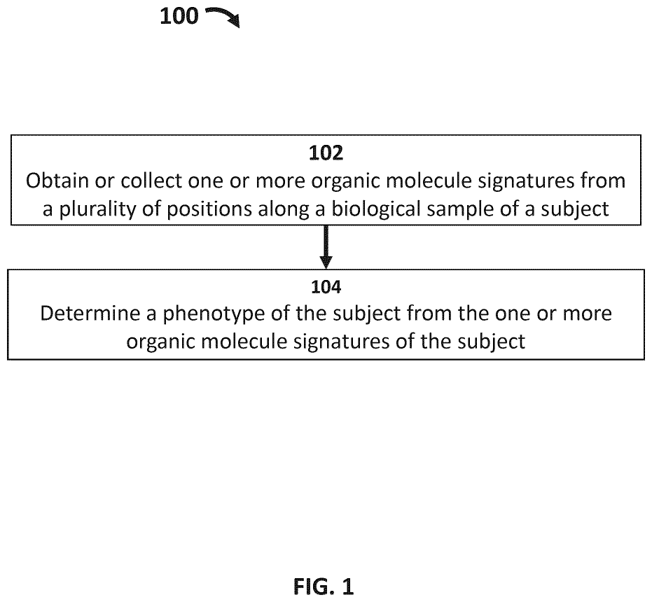

In some cases, the disclosure provided herein describes a method for classifying a phenotype of a subject 100, as seen in FIG. 1. In some instances, the method may comprise: obtaining, collecting, and/or sampling one or more organic molecule signatures from a plurality of positions along a biological sample of a subject 102; and determining a phenotype of the subject from the one or more organic molecule signatures of the subject 104. In some cases, the one or more organic molecule signatures may comprise a molecular signature of molecule with a mass-to-charge ratio of at least 50 Daltons (Da). In some cases, determining the phenotype of the subject may comprise: training a predictive model, as described elsewhere herein, with one or more organic molecule signatures and associated phenotype labels of a set of subjects different than the subject; and providing the subject's one or more organic molecule signatures to the trained predictive model and outputting the phenotype of the subject. In some cases, sampling may produce a set of data points of one or more organic molecule signatures at one or more positions of the plurality of positions on the biological sample. In some cases, the set of data points may be indicative of a dynamic biological response of a subject measured at the plurality of positions along the biological sample. A position of the biological sample where one or more organic molecule signatures are obtained, collected, and/or sampled, may correspond to a specific time of growth of the biological sample, e.g., an earlier or later point in time of the biological sample's growth. For example, in the case of a hair biological sample, the one or more positions of the hair biological sample may correspond to about 20-minute period of hair growth of a subject, where a segment of about 1 centimeter may correspond to a time period of about a month of growth of the subject.

In some cases, a first position of the plurality of positions may be adjacent to a second position of the plurality of positions. In some instances, the plurality of positions may be overlapping. In some cases, the plurality of positions may not overlap and may comprise distinct regions on the biological sample. In some cases, the plurality of positions may be separated by a pre-defined distance. In some instances, the distance may vary across the plurality of positions along the biological sample. In some instances, obtaining, collecting, and/or sampling may be performed along an axis of the biological sample beginning at a position of the axis corresponding to a position nearest to a root or beginning of a biological sample e.g., a position of the biological sample nearest a hair root, a position on a nail nearest the cuticle, a position towards a central axis of a cross section of a tooth, or any combination thereof. In some cases, the one or more organic molecule signature obtained at a position at the beginning of the biological sample may indicate and/or represent one or more organic molecule signatures at an earlier point in development of life for a subject.

In some cases, the plurality of positions comprises at least about 100, at least about 200, at least about 300, at least about 500, at least about 700, at least about 800, at least about 1000, at least about 1500, at least about 2000, at least about 2500, at least about 3000, at least about 3500, at least about 4000, at least about 4500, at least about 5000, at least about 5500, at least about 6000, at least about 6500, at least about 7000, at least about 7500, at least about 8000, at least about 8500, at least about 9000, at least about 9500, at least about 10000, or at least about 10000 positions on the biological sample.

In some cases, the one or more organic molecule signatures may comprise a molecular signature of a molecule with a mass-to-charge ratio of about 50 Da to about 500,000 Da. In some cases, the one or more organic molecule signatures may comprise a molecular signature of a molecule with a mass-to-charge ratio of about 50 Da to about 100 Da, about 50 Da to about 500 Da, about 50 Da to about 1,000 Da, about 50 Da to about 10,000 Da, about 50 Da to about 20,000 Da, about 50 Da to about 40,000 Da, about 50 Da to about 50,000 Da, about 50 Da to about 100,000 Da, about 50 Da to about 250,000 Da, about 50 Da to about 500,000 Da, about 100 Da to about 500 Da, about 100 Da to about 1,000 Da, about 100 Da to about 10,000 Da, about 100 Da to about 20,000 Da, about 100 Da to about 40,000 Da, about 100 Da to about 50,000 Da, about 100 Da to about 100,000 Da, about 100 Da to about 250,000 Da, about 100 Da to about 500,000 Da, about 500 Da to about 1,000 Da, about 500 Da to about 10,000 Da, about 500 Da to about 20,000 Da, about 500 Da to about 40,000 Da, about 500 Da to about 50,000 Da, about 500 Da to about 100,000 Da, about 500 Da to about 250,000 Da, about 500 Da to about 500,000 Da, about 1,000 Da to about 10,000 Da, about 1,000 Da to about 20,000 Da, about 1,000 Da to about 40,000 Da, about 1,000 Da to about 50,000 Da, about 1,000 Da to about 100,000 Da, about 1,000 Da to about 250,000 Da, about 1,000 Da to about 500,000 Da, about 10,000 Da to about 20,000 Da, about 10,000 Da to about 40,000 Da, about 10,000 Da to about 50,000 Da, about 10,000 Da to about 100,000 Da, about 10,000 Da to about 250,000 Da, about 10,000 Da to about 500,000 Da, about 20,000 Da to about 40,000 Da, about 20,000 Da to about 50,000 Da, about 20,000 Da to about 100,000 Da, about 20,000 Da to about 250,000 Da, about 20,000 Da to about 500,000 Da, about 40,000 Da to about 50,000 Da, about 40,000 Da to about 100,000 Da, about 40,000 Da to about 250,000 Da, about 40,000 Da to about 500,000 Da, about 50,000 Da to about 100,000 Da, about 50,000 Da to about 250,000 Da, about 50,000 Da to about 500,000 Da, about 100,000 Da to about 250,000 Da, about 100,000 Da to about 500,000 Da, or about 250,000 Da to about 500,000 Da. In some cases, the one or more organic molecule signatures may comprise a molecular signature of a molecule with a mass-to-charge ratio of about 50 Da, about 100 Da, about 500 Da, about 1,000 Da, about 10,000 Da, about 20,000 Da, about 40,000 Da, about 50,000 Da, about 100,000 Da, about 250,000 Da, or about 500,000 Da. In some cases, the one or more organic molecule signatures may comprise a molecular signature of a molecule with a mass-to-charge ratio of at least about 50 Da, about 100 Da, about 500 Da, about 1,000 Da, about 10,000 Da, about 20,000 Da, about 40,000 Da, about 50,000 Da, about 100,000 Da, or about 250,000 Da. In some cases, the one or more organic molecule signatures may comprise a molecular signature of a molecule with a mass-to-charge ratio of at most about 100 Da, about 500 Da, about 1,000 Da, about 10,000 Da, about 20,000 Da, about 40,000 Da, about 50,000 Da, about 100,000 Da, about 250,000 Da, or about 500,000 Da.

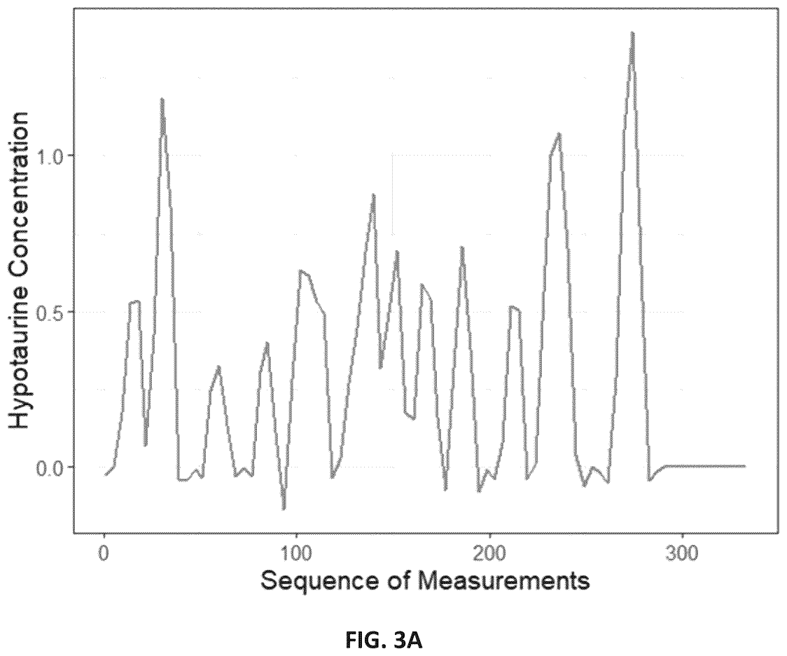

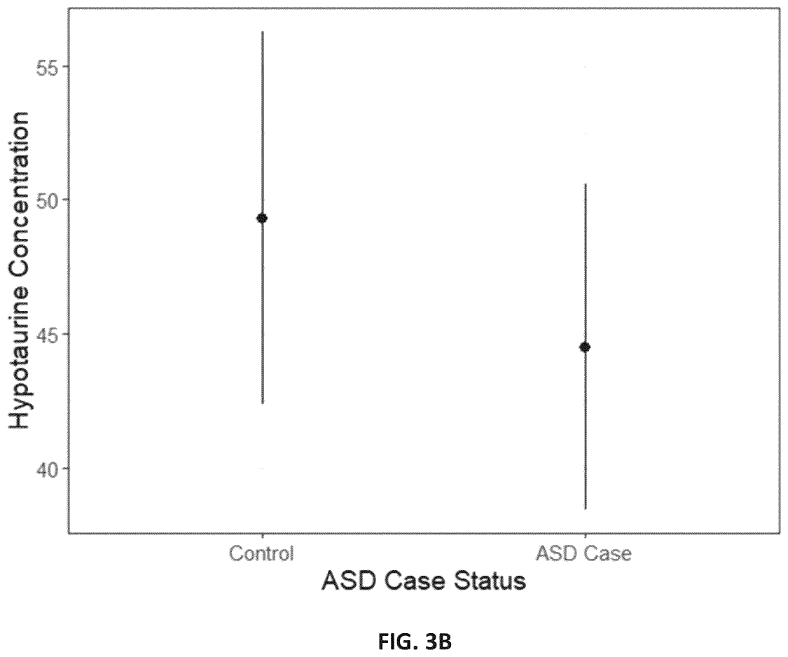

In some instances, the one or more organic molecule signatures may comprise one or more time-resolved organic molecule signatures, as shown in FIGS. 3A, 4A, 5A, 6A, 7A, and/or 8A. For example, the one or more organic molecule signatures may be determined at each position in a plurality of positions along the biological sample, described elsewhere herein. In some cases, the plurality of positions along the biological sample may be a plurality of positions along an axis (e.g., a linear and/or non-linear axis) on the biological sample. In some instances, the linear and/or non-linear axis may be on one or more surfaces of the biological sample. In some cases, a first position and a second position of the plurality of positions may spatially overlap or may be spatially separate and not overlap. In some instances, each position in the plurality of positions along the biological sample may provide a temporal dimension to the one or more organic molecule signatures that corresponds with periods of time and/or growth of a subject. In some cases, the temporal fluctuations in concentration of the one or more molecular signatures collected at the plurality of positions along the biological sample may provide a characterization and/or determination of a subject's phenotype with an accuracy of at least about 90%, at least about 92%, at least about 94%, at least about 96%, at least about 98%, or at least about 98%. In some cases, a geometric mean or average of the temporal organic molecule concentration may be calculated and used as a feature in characterizing and/or classifying a subject's phenotype.

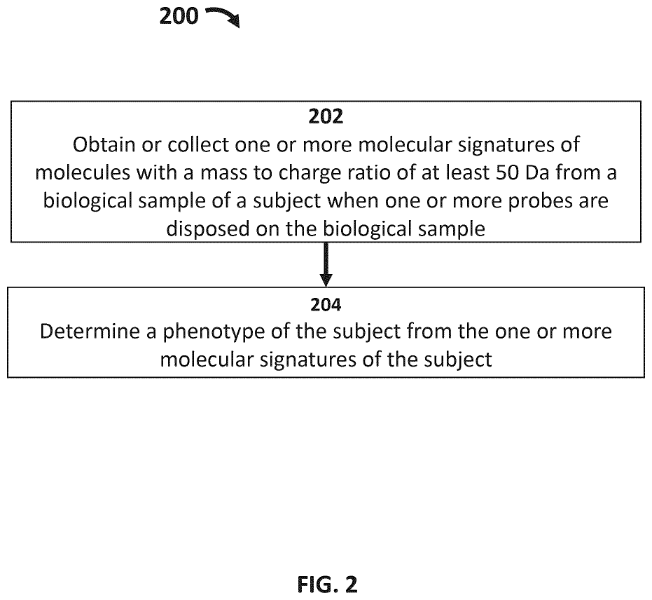

In some cases, the disclosure provided herein describes a method for classifying a phenotype of a subject 200, as seen in FIG. 2. In some cases, the method may comprise: obtaining or collecting one or more molecular signatures of molecules with a mass to charge ratio of at least 50 Da from a biological sample of a subject when one or more probes are disposed on the biological sample 202; and determining the phenotype of the subject from the one or more molecular signatures of the subject. In some cases, the one or more molecular signatures may be determined simultaneously 204. In some cases, the one or more molecular signatures may comprise one or more organic molecule signatures. In some cases, a light source may be used to obtain or collect the one or more molecular signatures from the one or more probes disposed on the biological sample. In some instances, the light source may comprise a laser, pulsed laser, continuous wave laser, or any combination thereof. In some cases, the one or more probes may comprise a binding moiety that is configured to bind to a protein. In some cases, a first probe of the one or more probes may comprising a binding moiety targeted to bind to a first protein and a second probe of the one or more probes may comprise a binding moiety targeted to bind to a second protein. In some instances, the first protein and the second protein may be positioned at a distance such that the first probe and the second probe are able to couple and form a probe complex. In some cases, a signaling molecule, e.g., a fluorescent molecule, may bind to the probe complex and may be detected by exciting the signaling molecule with the light source and detecting a signal therefrom. In some cases, detection of the fluorescent molecule and/or an associated intensity of the fluorescent molecule may provide a concentration and/or presence of a molecule of the one or more molecular signatures of the biological sample. In some cases, a composite index may be calculated and/or determined from the one or more molecular signatures of one or more subjects. In some instances, the composite index may be used to differentiate one or more groups or sets of subjects based on their classified and/or characterized phenotype. In some cases, the composite index may be determined by one or more computational methods. In some cases, the computational methods may comprise supervised discriminant analysis, canonical analysis, exploratory factor analysis, confirmatory factor analysis, partial least squares, partial least squares discriminant analysis, linear discriminant analysis, or any combination thereof. In some cases, determining the phenotype of the subject may comprise: training a predictive model, as described elsewhere herein, with one or more molecular signatures and associated phenotype labels of a set of subjects different than the subject; and providing the subject's one or more molecular signatures to the trained predictive model and outputting the phenotype of the subject.

In some instances, determining and/or classifying a phenotype of a subject may comprise processing the one or more molecular signatures acquired at one or more positions of the plurality of positions on the biological sample of the subject. In some cases, determining and/or classifying a phenotype of a subject from the one or more molecular signatures, e.g., the one or more molecular signatures obtained and/or acquired at one or more positions of the plurality of positions on the biological sample of the subject, as described elsewhere herein, may comprise processing the one or more molecular signatures obtained and/or acquired at the one or more positions of the plurality of positions of the biological sample to generate one or more features of the one or more molecular signatures. In some cases, computational analysis and/or processing may comprise determining a difference between one or more molecular signatures across the biological sample e.g., one or more molecular signatures obtained and/or acquired at one or more adjacent positions of the plurality of positions along the axis of the biological sample, as described elsewhere herein. In some cases, one or more adjacent positions of the plurality of positions on the biological sample may comprise a temporal relationship. For example, one or more molecular signatures obtained and/or collected at a first position of the biological sample may comprise and/or be associated with a value of a first point in time, and one or more molecular signatures obtained and/or collected second position adjacent to the first position on the biological sample may comprise and/or be associated with a value of a second point in time different than the first point in time. In some cases, the one or more molecular signatures between a first, second, and/or another position of the plurality of positions may provide a temporal signature of one or more molecular signatures. In some cases, temporal dynamics of the one or more molecular temporal signatures may be processed and/or analyzed. In some cases, the one or more molecular signatures may be processed and/or analyzed by techniques and/or methods of dimensionality reduction e.g., by independent component analysis (ICA) and/or principal component analysis (PCA), non-negative matrix factorization (NNMF), unsupervised dimensionality reduction, supervised dimensionality reduction, or any combination thereof, of the one or more molecular signatures and/or one or more features of the one or more molecular signatures, described elsewhere herein. In some instances, the one or more temporal dynamics of the one or more molecular signatures may be process and/or analyzed with recurrence quantification analysis (RQA) to extract features descriptive of the one or more temporal dynamics of the one or more molecular signatures and/or to extract features of the dimensionally reduced one or more molecular signatures, described elsewhere herein. In some cases, processing and/or analyzing the temporal dynamics of the one or more molecular signatures may result in one or more features of the temporal dynamics of the one or more molecular signatures. In some cases, the one or more features may comprise recurrence rates, recurrence time (RT), determinism, Lmax, mean diagonal length (MDL), maximum diagonal length, divergence, Shannon entropy in diagonal length, trend in recurrences, laminarity, trapping time (T), maximum vertical line length, Shannon entropy in vertical line lengths, mean recurrence time, Shannon entropy in recurrence times, number of the most probable recurrences, or any combination thereof features.

RQA may measure variability in temporal and/or time-dependent domains and/or characteristics of the one or more molecular signatures obtained for one or more positions of the plurality of positions of the biological sample, as described elsewhere herein. RQA may involve the estimation of features, described elsewhere herein, that describe periodic properties of one or more temporal and/or time-dependent aspects of the one or more molecular signatures.

Methods and features of RQA are described, for example, by Webber et al. in “Simpler Methods Do It Better: Success of Recurrence Quantification Analysis as a General Purpose Data Analysis Tool,” Physics Letters A 373, 3753-3756 (2009) and by Marwan et al. in “Recurrence Plots for the Analysis of Complex Systems,” Physics Reports 438, 237-239 (2007), the contents of each of which are herein incorporated by reference in their entirety. In some embodiments, the one or more time-dependent molecular signatures may be analyzed with other analytical methods, such as Fourier transformations, wavelet analysis, cosinor analysis, or any combination thereof. Such techniques may be applied to derive similar metrics, including spectral analysis of frequency components and their associated power of the one or more molecular signatures. These metrics and associated derivative measures may be used in place of and/or in addition to the features derived from RQA to analyze the one or more time-dependent molecular signatures obtained from biological samples to provide, determine, classify, and/or characterize, one or more phenotypes of a subject and/or phenotypes of a plurality of subjects.

RQA may comprise determining, constructing, and/or displaying of recurrence plots, which visualize and/or analyze dynamical temporal structures of the one or more molecular signatures. Such recurrence plots may illustrate phasic processes in sequential measurements by plotting a given sequence against a time-lagged derivation of that sequence. From the one dimensional molecular signature measured from the hair shaft, additional dimensions may be computationally derived to embed the molecular signature in a higher dimensional space referred to as a phase portrait, where t refers to the values of the original molecular signature, and dimensions (t+τ) and (t+2τ) may be derived from lagging the original molecular signature time series by interval τ. Subsequent analyses may then be undertaken on the embedded phase portrait to construct recurrence plots and recurrence quantification analysis. A recurrence quantification plot may be derived from the phase portrait through the application of a threshold function to each point in the phase portrait; on the corresponding recurrence plot, consisting of a square binary matrix, typically represented as white or black space, a given point is assigned a value of 1 at each temporal interval wherein another point in the phase-portrait shares the spatial limits of the assigned threshold boundary. The RQA method may be applied to the recurrence plot to examine the interval of delay between states in a given system, with a black point reflecting the temporal interval when a system revisits the same state. Periodic processes, where a system successively reiterates a given pattern of states, may manifest in a recurrence plot as diagonal black lines, whereas periods of stability will manifest as square structures, spurious repetitions as black dots, and unique events as white space.

In some embodiments, the recurrence plots may be constructed for one or more molecular signatures (e.g., to visualize an interactive periodic pattern of two or more molecular signatures; this can be referred to as cross-recurrence quantification analysis, or joint-recurrence quantification analysis). In some embodiments, the recurrence plots are constructed for a combination of three or more molecular signatures.

In some embodiments, the data analysis may include analyzing the recurrence plots to obtain a set of features associated with the recurrence plots. The features, which interchangeably can be termed “rhythmicity features,” or “dynamic features,” may provide a quantitative measure describing the periodicity, predictability, and transitivity present in the one or more molecular signatures. The features may be selected from a set including recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof, as described elsewhere herein.

In some embodiments, the data analysis may further comprise providing the obtained set of features as an input to one or more trained predictive models and/or one or more machine learning algorithms. In some embodiments, the one or more trained predictive models and/or the one or more machine learning algorithms may comprise a predictive computational algorithm to obtain a probability for one or more subjects having a phenotype. In some embodiments, the predictive model and/or machine learning computational algorithm may perform the following calculation:

$$p ( subject ) = \frac{1}{1 + {e}^{- ( α + {β}_{1} {x}_{1} + ⋯ + {β}_{k} {x}_{k} )}}$$

where p(subject) is the probability that a subject has a phenotype, e is Euler's number, α is a calculated parameter associated with the probability that the subject has the phenotype when β₁x₁+ . . . +βₖxₖ equals to zero, x₁, . . . , xₖ corresponds to a value derived for each feature in the set of features, described elsewhere herein, the set of features including features from 1 through k, and β₁, . . . , βₖ corresponds to a weight parameter associated with each feature in the set of features including features from 1 through k.

The weight parameters β₁, . . . , βₖ may be defined based on predictive model and/or machine learning algorithm training. The probability p(subject) may be provided as a number ranging from 0 to 1, where 1 corresponds to a 100% probability that the subject has a phenotype.

In some embodiments, the data analysis may include applying a threshold to the obtained probability p(subject). If the obtained probability p(subject) is above the predetermined threshold, the subject may be evaluated as having the phenotype. If the obtained probability is below the threshold, the subject may be evaluated as not having the phenotype. In some embodiments, the threshold may be between about 0.3 and 0.6 (e.g., the predetermined threshold is about 0.3, about 0.35, about 0.4, about 0.45, about 0.5, about 0.55, or about 0.6). The value assigned for a probabilistic threshold may be predetermined or estimated during the training of the predictive model and/or machine learning algorithm through the use of receiver-operating-characteristic (ROC) charts, with the optimal threshold used corresponding to the value which yields the maximum area-under-the-curve (AUROC). In some embodiments, the obtained probability may be expressed in terms of associated odds (e.g., odds ratio (OR)), which may be derived from a probability such that OR=p/(1−p)). For example, the evaluation may comprise evaluating odds that the subject has the biological condition.

Although the above operations show each of the methods or sets of operations in accordance with embodiments, many variations may be performed. The operations may be completed in a different order. Operations may be added or omitted. Some of the operations may comprise sub-operations. Many of the operations may be repeated as often as beneficial.

One or more of the operations of each of the methods or sets of operations may be performed with circuitry as described herein, for example, one or more of the processor or logic circuitry such as programmable array logic for a field programmable gate array. The circuitry may be programmed to provide one or more of the operations of each of the methods or sets of operations, and the program may comprise program instructions stored on a computer readable memory or programmed operations of the logic circuitry such as the programmable array logic or the field programmable gate array, for example.

### Systems

The disclosure provided herein describes systems to execute and/or implement the methods of the disclosure, as described elsewhere herein. In some cases, the systems may comprise a system for classifying a phenotype of a subject, comprising: one or more processors and memory storing one or more programs for execution by the one or more processors, the one or more programs comprising instructions to: (i) obtain, collect, and/or receive one or more organic molecule signatures of a biological sample of the subject from a plurality of positions along the biological sample; and (ii) determine the phenotype of the subject from the one or more organic molecule signatures of the subject. In some cases, the phenotype of the subject may comprise a molecular phenotype, physiologic phenotype, a behavior phenotype, a disease phenotype, a health phenotype, an exposure phenotype, one or more upregulated or downregulated physiological pathways, pharmaceutical response, nutraceutical response, presence of exogenous compounds, presence of endogenous compounds, presence of inflammation, or any combination thereof. In some cases, the disease phenotype may comprise autism spectrum disorder (ASD), attention-deficit/hyperactivity disorder, amyotrophic lateral sclerosis (ALS), cancer, or any combination thereof. In some cases, the exogenous compounds may comprise nicotine, melamine, or a combination thereof. In some instances, the endogenous compounds may comprise metabolites, signaling molecules, or a combination thereof. The signaling molecules may comprise hypotaurine. In some cases, the endogenous metabolites may comprise creatinine. In some instances, the nutraceutical may comprise agmatine. In some instances, the pharmaceutical may comprise a pharmaceutical to treat heart burn, acid reflux, peptic ulcers, or any combination thereof. In some cases, the pharmaceutical may comprise Betazole. In some instances, the exposure phenotype may comprise exposure of the subject to environmental chemicals. In some cases, the biological sample may comprise hair, teeth, fingernails, toenails, or any combination thereof.

In some cases, the one or more organic molecule signatures may comprise molecular signature of a molecule with a mass-to-charge ratio of at least 50 Da, or a mass-to-charge ratio as described elsewhere herein. In some cases, the one or more organic molecule signatures may comprise one or more time-resolved organic molecule signatures.

In some instance, the system may comprise a light source configured to obtain or collect the one or more organic molecule signatures from the plurality of positions along the biological sample. In some cases, the light source may comprise a laser, pulsed laser, continuous wave laser, or any combination thereof. In some cases, the one or more organic molecule signatures may be obtained or collected by conducting MALDI-ToF MS, LAESI, TIMS, ToF mass spectrometry, protein fluorescence assays, or any combination thereof, with the biological sample.

In some cases, the system may comprise instructions of determining a change and/or a response of a subject's phenotype when the subject is administered a pharmaceutical and/or nutraceutical. In some cases, a subject's phenotype may be used to inform how to adjust e.g., change the pharmaceutical and/or nutraceutical compound entirely, add to the pharmaceutical and/or nutraceutical compound, identify a new pharmaceutical and/or nutraceutical compound to administer to the subject, change the dosing regimen of the pharmaceutical nutraceutical compound, or any combination thereof. In some instances, the pharmaceutical and/or nutraceutical administered may ameliorate a disease or one or more symptoms of a disease and/or condition of the subject.

In some cases, the instructions of determine the phenotype of the subject may comprise: train a predictive model, as described elsewhere herein, with one or more organic molecule signatures and associated phenotype labels of a set of subjects different than the subject; and provide the subject's one or more organic molecular signatures to the trained predictive model and output the phenotype of the subject.

The disclosure provided herein describes systems to execute and/or implement the methods of the disclosure, as described elsewhere herein. In some cases, the systems comprise a system for classifying a phenotype of the subject, comprising: one or more processors and memory storing one or more programs for execution by the one or more processors, the one or more programs comprising instructions to: (i) obtain or collect one or more molecular signatures of molecules with a mass to charge ratio of at least 50 Da from a biological sample of a subject when one or more probes are disposed on the biological sample; and (ii) determine the phenotype of the subject from the one or more molecular signatures of the subject. In some cases, the phenotype of the subject may comprise a molecular phenotype, physiologic phenotype, a behavior phenotype, a disease phenotype, a health phenotype, an exposure phenotype, one or more upregulated or downregulated physiological pathways, pharmaceutical response, nutraceutical response, presence of exogenous compounds, presence of endogenous compounds, presence of inflammation, or any combination thereof. In some cases, the disease phenotype may comprise autism spectrum disorder (ASD), attention-deficit/hyperactivity disorder, amyotrophic lateral sclerosis (ALS), or any combination thereof. In some cases, the exogenous compounds may comprise nicotine, melamine, or any combination thereof. In some instances, the endogenous compounds may comprise metabolites, signaling molecules, or any combination thereof. The signaling molecules may comprise hypotaurine. In some cases, the endogenous metabolites may comprise creatinine. In some instances, the nutraceutical may comprise agmatine. In some instances, the pharmaceutical may comprise a pharmaceutical to treat heart burn, acid reflux, peptic ulcers, or any combination thereof. In some cases, the pharmaceutical may comprise Betazole. In some instances, the exposure phenotype may comprise exposure of the subject to environmental chemicals. In some cases, the biological sample may comprise hair, teeth, fingernails, toenails, or any combination thereof.

In some cases, the system may comprise a light source that may be used to obtain or collect the one or more molecular signatures from the one or more probes disposed on the biological sample. In some instances, the light source may comprise a laser, pulsed laser, continuous wave laser, or any combination thereof. In some cases, the one or more probes may comprise a binding moiety that is configured to bind to a protein. In some cases, a first probe of the one or more probes may comprising a binding moiety targeted to bind to a first protein and a second probe of the one or more probes may comprise a binding moiety targeted to bind to a second protein. In some instances, the first protein and the second protein may be positioned at a distance such that the first probe and the second probe are able to couple and form a probe complex. In some cases, a signaling molecule, e.g., a fluorescent molecule, may bind to the probe complex and may be detected by the light source. In some cases, detection of the fluorescent molecule and/or an associated intensity of the fluorescent molecule may provide a concentration of a molecule of the one or more molecular signatures of the biological sample. In some cases, the instructions of determine the phenotype of the subject may comprise: train a predictive model, as described elsewhere herein, with one or more molecular signatures and associated phenotype labels of a set of subjects different than the subject; and provide the subject's one or more molecular signatures to the trained predictive model and output the phenotype of the subject.

In some cases, the system may comprise instructions of determining a change and/or a response of a subject's phenotype when the subject is administered a pharmaceutical and/or nutraceutical. In some cases, a subject's phenotype may be used to inform how to adjust e.g., change the pharmaceutical and/or nutraceutical compound entirely, add to the pharmaceutical and/or nutraceutical compound, identify a new pharmaceutical and/or nutraceutical compound to administer to the subject, change the dosing regimen of the pharmaceutical nutraceutical compound, or any combination thereof. In some instances, the pharmaceutical and/or nutraceutical administered may ameliorate a disease or one or more symptoms of a disease and/or condition of the subject.

### Computer Systems

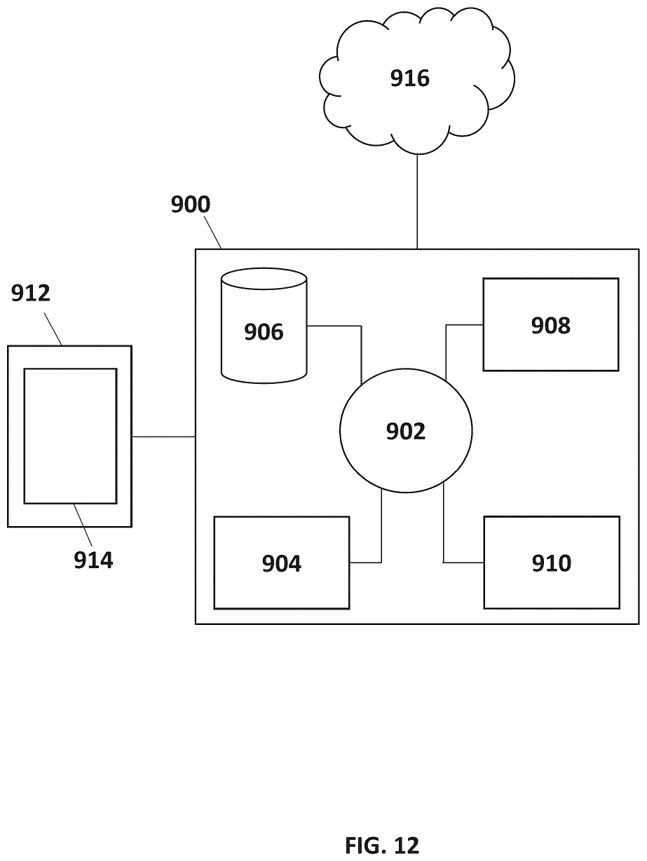

FIG. 12 shows a computer system 900 suitable for implementing the methods of the disclosure, described elsewhere herein. In some cases, the computer system may process one or more signals of the collected and/or obtained one or more molecular signatures, as described elsewhere herein, implementing and/or training one or more machine learning algorithms and/or one or more predictive models, described elsewhere herein. The computer system 900 may process various aspects of data and/or information of the present disclosure, such as, for example, subjects' one or more molecular signatures (e.g., one or more organic molecule signatures) of a biological sample, features derived from the one or more molecular signatures, features derived from average and/or geometrical mean of a signal (e.g., concentration) of the one or more organic molecule signatures collected and/or obtained across a plurality of positions of a biological sample, features derived from one or more molecular signatures obtained from one or more probes disposed on e.g., a surface of a biological sample, subject phenotype characterizations, or any combination thereof. The computer system 900 may be an electronic device. The electronic device may be a mobile electronic device, desktop computer device, laptop device, server, cloud computing platform, or any combination thereof.

The computer system 900 may comprise a central processing unit (CPU, also “processor” and “computer processor” herein) 902, which may be a single core or multi core processor, or a plurality of processor for parallel processing. The computer system 900 may further comprise memory or memory locations 904 (e.g., random-access memory, read-only memory, flash memory), electronic storage unit 906 (e.g., hard disk), communications interface 908 (e.g., network adapter) for communicating with one or more other devices, and peripheral devices 910, such as cache, other memory, data storage and/or electronic display adapters. The memory 904, storage unit 906, interface 908, and peripheral devices 910 may be in communication with the CPU 902 through a communication bus (solid lines), such as a motherboard. The storage unit 906 may be a data storage unit (or a data repository either a local and/or networked data repository) for storing data. The computer system 900 may be operatively coupled to a computer network (“network”) 916 with the aid of the communication interface 908. The network 916 may be the Internet, an internet and/or extranet, or an intranet and/or extranet that is in communication with the Internet. The network 916 may, in some case, be a telecommunication and/or data network. The network 916 may include one or more computer servers, which may enable distributed computing, such as cloud computing. The network 916, in some cases with the aid of the computer system 900, may implement a peer-to-peer network, which may enable devices coupled to the computer system 900 to behave as a client or a server.

The CPU 902 may execute a sequence of machine-readable instructions, which may be embodied in a program or software. The instructions may be directed to the CPU 902, which may subsequently program or otherwise configure the CPU 902 to implement methods of the disclosure described elsewhere herein. Examples of operations performed by the CPU 902 may include fetch, decode, execute, and writeback.

The CPU 902 may be part of a circuit, such as an integrated circuit. One or more other components of the system 900 may be included in the circuit. In some cases, the circuit may be an application specific integrated circuit (ASIC).

The storage unit 906 may store files, such as drivers, libraries, and/or saved programs. The storage unit 906 may store subjects' one or more molecular signatures (e.g., one or more organic molecule signatures) of a biological sample, features derived from the one or more molecular signatures, features derived from average and/or geometrical mean of a concentration of the one or more organic molecule signatures collected and/or obtained across a plurality of positions of a biological sample, features derived from one or more molecular signatures obtained from one or more probes disposed on a surface of a biological sample, subject phenotype characterizations, or any combination thereof. The computer system 900, in some cases, may include one or more additional data storage units that are external to the computer system 900, such as located on a remote server that is in communication with the computer system 900 through an intranet or the internet.

Methods as described herein may be implemented by way of machine (e.g., computer processor) executable code stored on an electronic storage location of the computer device 900, such as, for example, on the memory 904 and/or electronic storage unit 906. The machine executable and/or machine-readable code may be provided in the form of software. During use, the code may be executed by the processor 902. In some instances, the code may be retrieved from the storage unit 906 and stored on the memory 904 for ready access by the processor 902. In some instances, the electronic storage unit 906 may be precluded, and machine-executable instructions may be stored on memory 904.

The code may be pre-compiled and configured for use with a machine having a processor adapted to execute the code or may be compiled during runtime. The code may be supplied in a programming language that may be selected to enable the code to be executed in a pre-complied or as-compiled fashion.

Aspects of the systems and methods provided herein, such as the computer system 900, may be embodied in programming. Various aspects of the technology may be thought of a “product” or “articles of manufacture” typically in the form of a machine (or processor) executable code and/or associated data that is carried on or embodied in a type of machine-readable medium. Machine-executable code may be stored on an electronic storage unit, such memory (e.g., read-only memory, random-access memory, flash memory) or a hard disk. “Storage” type media may include any or all of the tangible memory of a computer, processor, and/or associated modules thereof, such as various semiconductor memories, tape drives, disk drives and the like, which may provide non-transitory storage at any time for the software programming. All or portions of the software may at times be communicated through the Internet, intranet, or a combination thereof, and/or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, term such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media may include, for example, optical or magnetic disks, such as any of the storage devices in any computer(s) or the like, such as may be used to implement the databases, etc. Volatile storage media may include dynamic memory, such as main memory of such a computer platform. Tangible transmission media includes coaxial cables, copper wire, fiber optics, or any combination thereof, e.g., the wires that comprise a bus within a computer device. Carrier-wave transmission media may take the form of electric or electromagnetic signals, or acoustic or light waves such as those generated during radio frequency (RF) and infrared (IR) data communications. Common forms of computer-readable media therefor include for example: a floppy disk, a flexible disk, hard disk, magnetic tape, any other magnetic medium, a CD-ROM, DVD or DVD-ROM, any other optical medium, punch cards paper tape, any other physical storage medium with pattern of holes, a RAM, a ROM, a PROM and EPROM, a FLASH-EPROM, any other memory chip or cartridge, a carrier wave transporting data or instructions, cables or links transporting such a carrier wave, any combination hereof, or any other medium from which a computer may read programming code and/or data. Many of these forms of computer readable media may be involved in carrying one or more sequences of one more instruction to a processor for execution.

The computer system may include or be in communication with an electronic display 912 that may comprise a user interface (UI) 914 for viewing the one or more molecular signatures and/or phenotypic characterization prediction of a subject based on the one or more molecular signatures (e.g., one or more organic molecule signatures) of the subject's biological sample. Examples of UI's include, without limitation, a graphical user interface (GUI) and web-based user interface.

### Predictive Models and Machine Learning Algorithms

Methods and/or systems of the disclosure can process, analyze, and/or classify one or more molecular signatures and/or one or more features of the temporal dynamics of the one or more molecular signatures of a biological sample to determine a phenotype, as described elsewhere herein. In some cases, the processing, analyzing, and/or classifying one or more molecular signatures and/or one or more features of the one or more molecular signatures may be conducted by way of one or more machine learning algorithms and/or one or more predictive models with instructions provided to one or more processors as described elsewhere herein. For example, one or more machine learning algorithms and/or predictive models may process one or more, or two or more features (i.e, a plurality of features) of the one or more molecular signatures, described elsewhere herein.

In some cases, the subject's and/or plurality of subjects' phenotypes may be determined and/or predicted with one or more machine learning algorithms and/or one or more predictive models with a sensitivity of at least about 70%, at least about 75%, at least about 80%, at least about 85%, or at least about 90%.

In some cases, the subject's and/or plurality of subjects' phenotypes may be determined and/or predicted with one or more machine learning algorithms and/or one or more predictive models with a sensitivity of up to about 70%, up to about 75%, up to about 80%, up to about 85%, or up to about 90%.

In some cases, the subject's and/or plurality of subjects' phenotypes may be determined and/or predicted with one or more machine learning algorithms and/or one or more predictive models with a specificity of at least about 70%, at least about 75%, at least about 80%, at least about 85%, or at least about 90%.

In some cases, the subject's and/or plurality of subjects' phenotypes may be determined and/or predicted with one or more machine learning algorithms and/or one or more predictive models with a specificity of up to about 70%, up to about 75%, up to about 80%, up to about 85%, or up to about 90%.

In some cases, the subject's and/or plurality of subjects' phenotypes may be determined and/or predicted with one or more machine learning algorithms and/or one or more predictive models with a positive predictive value of at least about 70%, at least about 75%, at least about 80%, at least about 85% or at least about 90%.

In some cases, the subject's and/or plurality of subjects' phenotypes may be determined and/or predicted with one or more machine learning algorithms and/or one or more predictive models with a positive predictive value of up to about 70%, up to about 75%, up to about 80%, up to about 85%, or up to about 90%.

In some cases, the subject's and/or plurality of subjects' phenotypes may be determined and/or predicted with one or more machine learning algorithms and/or one or more predictive models with a negative predictive value of at least about 70%, at least about 75%, at least about 80%, at least about 85%, or at least about 90%.

In some cases, the subject's and/or plurality of subjects' phenotypes may be determined and/or predicted with one or more machine learning algorithms and/or one or more predictive models with a negative predictive value of up to about 70%, up to about 75%, up to about 80%, up to about 85%, or up to about 90%.

In some cases, the subject's and/or plurality of subjects' phenotypes may be determined and/or predicted with one or more machine learning algorithms and/or one or more predictive models with an Area Under the Receiver Operating Characteristic Curve(AUROC) of at least about 0.65, at least about 0.70, at least about 0.75, at least about 0.80, at least about 0.82, at least about 0.84, at least about 0.86, at least about 0.88, or at least about 0.90.

In some cases, the subject's and/or plurality of subjects' phenotypes may be determined and/or predicted with one or more machine learning algorithms and/or one or more predictive models with an Area Under the Receiver Operating Characteristic (AUROC) of up to about 0.65, up to about 0.70, up to about 0.75 up to about 0.80, up to about 0.82, up to about 0.84, up to about 0.86 up to about 0.88, or up to about 0.90.

An algorithm and/or predictive model can be implemented by way of software upon execution by the central processing unit 902. In some cases, the predictive model may comprise a machine learning predictive model. In some cases, the machine learning predictive model may comprise one or more statistical, machine learning, or artificial intelligence algorithms. Examples of utilized algorithms, machine learning algorithms, and/or predictive models may include a support vector machine (SVM), a naïve Bayes classification, a random forest, a neural network (such as a deep neural network (DNN)), a recurrent neural network (RNN), a deep RNN, a long short-term memory (LSTM) recurrent neural network (RNN), decision tree algorithm, unsupervised clustering algorithm, a supervised clustering algorithm, a regression algorithm, a gradient-boosting algorithm (e.g., a gradient-boosting implementation of a machine learning algorithm and/or predictive model such as a gradient-boosted decision trees), a gated recurrent unit (GRU), supervised learning algorithm, unsupervised learning algorithm, statistical, deep-learning algorithm for classification and regression, or any combination thereof. In some cases, the recurrent neural network may comprise units which can be LSTM units and/or GRU. In some cases, the predictive model and/or the machine learning algorithm may comprise an ensemble of one or more predictive models and/or machine learning algorithms. In some cases, the one or more predictive models and/or one or more machine learning algorithms may be arranged in parallel, e.g., a first one or more predictive models and/or one or more machine learning algorithms and a second one or more predictive models and/or one or more machine learning algorithms may provide an output to a third one or more predictive models and/or one or more machine learning algorithms that then renders a determine of a phenotype of one or more subjects.

The machine learning algorithm and/or predictive model may likewise involve the estimation of ensemble models, comprised of multiple predictive models, and utilize techniques such as gradient boosting, for example in the construction of gradient-boosting decision trees. The machine learning predictive model may be trained using one or more training datasets corresponding to a one or more subjects' data. In some embodiments, the one or more training datasets may comprise one or more molecular signatures, one or more features of the one or more molecular signatures, and corresponding phenotype characterization of one or more subjects and/or one or more groups of one or more subjects' one or more molecular signatures, one or more features of the one or more molecular signatures, or a combination thereof. In some cases, one or more machine learning algorithms and/or one or more predictive models may be trained with one or more subjects' (e.g., one or more training subjects') one or more molecular signatures and/or features of the one or more molecular signatures acquired over a plurality of positions of a corresponding one or more biological samples of the one or more subjects, as described elsewhere herein.

In some cases, the disclosure describes a method of training an untrained or partially untrained machine learning algorithm or predictive model, comprising: at a computer system having one or more processors, and memory storing one or more programs for execution by the one or more processors: collecting or obtaining one or more molecular signatures of a biological sample of a plurality of training subjects, wherein a first subset of training subjects in the plurality of training subjects have a first phenotype associated with the one or more molecular signatures and a second subset of training subjects in the plurality of training subjects have a second phenotype associated with the one or more molecular signatures; and training the untrained or partially untrained machine learning algorithm or predictive model with the one or more molecular signatures of the plurality of training subjects and the corresponding first phenotype and second phenotype associated with the one or more molecular signatures thereby producing or generating a trained predictive model. In some cases, the one or more molecular signatures of the subject, the plurality of training subjects, or a combination thereof may comprise one or more organic molecule signatures. In some cases, the untrained or partially untrained machine learning algorithm and/or predictive model may be further trained on one or more signals and/or one or more images of a fluorescent marker of one or more protein probes bound to the biological sample.

In some cases, the disclosure describes a method for training a machine learning model or a predictive model, comprising: at a computer system, described elsewhere herein, having one or more processors, and memory storing one or more programs for execution by the one or more processors: (a) for each respective training subject in a plurality of training subjects, wherein a first subset of training subjects in the plurality of training subjects have a first phenotype, described elsewhere herein, associated with a first one or more features of a first one or more molecular signature and a second subset of training subjects in the plurality of training subjects have a second phenotype associated with a second one or more features of a second one or more molecular signature: (i) sampling, obtaining, and/or acquiring each respective position in a plurality of positions on a biological sample of the training subject, thereby obtaining a plurality of molecular signatures at a plurality of positions on the biological sample; (ii) analyzing and/or processing the one or more molecular signatures sampled, obtained, and/or acquired at the plurality of positions to determine and/or derive one or more feature of the one or more molecular signatures at the plurality of positions on the biological sample; and (b) training an untrained or partially untrained machine learning algorithm and/or predictive model with (i) the corresponding one or more features of each training subject in the plurality of training subjects, and (ii) the corresponding phenotype of each training subject in the plurality of training subjects, selected from among the first phenotype and the second phenotype, thereby obtaining a trained model that provides an indication as to whether a test subject has the first phenotype associated one or more of the one or more features of the one or more molecular signatures acquired at the plurality of positions of a biological sample of the test subject. In some cases, the machine learning model and/or the predictive model may be trained on one or more organic molecule signatures, described elsewhere herein. In some instances, a value of the one or more features of the subject may be provided to the trained machine learning algorithm and/or predictive model as an input where the machine learning algorithm and/or the predictive model may provide an output of a phenotype type of the subject.

In some cases, the trained one or more machine learning algorithms and/or the trained one or more predictive models may be configured to process and/or analyze one or more, and/or two or more features of the one or more molecular signatures, as described elsewhere herein, to e.g., determine and/or predict a phenotype of a subject and/or a plurality of subjects. In some cases, the trained one or more machine learning algorithms and/or the trained one or more predictive models may be configured to process and/or analyze one or more organic signatures, described elsewhere herein.

In some cases, the trained one or more predictive model(s) and/or trained one or more machine learning algorithm(s) may comprises a plurality of parameters, where the term “parameter” refers to any coefficient and/or any value of an internal or external element (e.g., a weight and/or a hyperparameter) in the one or more predictive model(s) and/or one or more machine learning algorithm(s) (e.g., where the one or more predictive model and/or one or more machine learning algorithm(s) are a regressor and/or a classifier) that can affect (e.g., modify, tailor, and/or adjust) one or more inputs, outputs, and/or functions in the one or more predictive model(s) and/or one or more machine learning algorithm(s). For example, in some embodiments, a parameter of a model and/or a machine learning algorithm may refer to any coefficient, weight, and/or hyperparameter that can be used to control, modify, tailor, and/or adjust the behavior, learning, and/or performance of the predictive model and/or the machine learning algorithm. In some instances, a parameter may be used to increase or decrease the influence of an input (e.g., a feature) of a predictive model and/or a machine learning algorithm. For example, a parameter may be used to increase or decrease the influence of a node (e.g., of a neural network), where the node includes one or more activation functions. Assignment of parameters to specific inputs, outputs, and/or functions of a predictive model and/or a machine learning algorithm may not be limited to any one paradigm for a given predictive model and/or machine learning algorithm but can be used in any suitable predictive model and/or machine learning algorithm for a desired performance. In some cases, a parameter may comprise a fixed value. In some instances, a value of a parameter may be manually and/or automatically adjustable. In some cases, a value of a parameter may be modified by a validation and/or training process for a predictive model and/or a machine learning algorithm (e.g., by error minimization and/or back propagation methods). In some embodiments, a predictive model and/or a machine learning algorithm of the present disclosure may comprise a plurality of parameters. In some embodiments, the plurality of parameters associated with a predictive model and/or a machine learning algorithm (e.g., an untrained, partially trained, and/or fully trained model) may comprise n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. In some cases, n may be between 10,000 and 1×10⁷, between 100,000 and 5×10⁶, or between 500,000 and 1×10⁶.

Training datasets may be generated from, for example, one or more cohorts of subjects having a common one or more features, e.g., molecular signatures, one or more features of the one or more molecular signatures, or a combination thereof, and phenotype (e.g., labels). Training datasets may comprise a set of features. Labels of the training data (e.g., the one or more molecular signatures and/or features of the one or more molecular signatures) of one or more subjects may comprise a phenotype of the subject (e.g., patient).

Features utilized to train one or more predictive models and/or one or more machine learning algorithms and/or provided as an input to the one or more trained predictive models and/or the one or more trained machine learning algorithms may comprise subject demographic information derived from electronic medical records (EMR), subject medical observations, or any combination thereof. Features may comprise clinical characteristics such as, for example, certain ranges or categories of dynamic molecular signatures. Features utilized to train one or more predictive models and/or one or more machine learning algorithms and/or provided as an input to the one or more trained predictive models and/or the one or more trained machine learning algorithms may comprise subject information such as subject age, subject medical history, other medical conditions, current or past medications taken by a subject, time since the last observation and/or evaluation of the subject, or any combination thereof. In some cases, the one or more predictive models and/or the one or more machine learning algorithms may comprise an algorithm architecture comprising a neural network with a set of input features e.g., subject vital signs and other subject health-based measurements, subject medical history, and/or subject demographics. For example, a set of features collected from a given subject at a given time point may collectively serve as a signature, which may be indicative of a phenotype of the subject at the given time point.

In some cases, ranges of molecular signature(s) data and/or other health measurements of one or more subjects may be expressed as a plurality of disjoint continuous ranges of continuous measurement values. In some instances, categories of molecular signatures and other health measurements may be expressed as a plurality of disjoint sets of measurement values (e.g., {“high”, “low”}, {“high”, “normal”}, {“low”, “normal”}, {“high”, “borderline high”, “normal”, “low”}, etc.). Clinical characteristics may also comprise clinical labels indicating the patient's health history, such as a prior determination, classification, and/or diagnosis of a phenotype, a previous administration of a clinical treatment (e.g., a drug, a surgical treatment, chemotherapy, radiotherapy, immunotherapy, etc.), behavioral factors, other health status (e.g., hypertension or high blood pressure, hyperglycemia or high blood glucose, hypercholesterolemia or high blood cholesterol, history of allergic reaction or other adverse reaction, etc.), or any combination thereof.

Clinical outcomes may include a temporal characteristic associated with the presence, absence, diagnosis, determination, and/or prognosis of a phenotype of the subject. For example, temporal characteristics may be indicative of the subject having had a classification, determination, and/or diagnosis of a phenotype within a certain period of time after a previous clinical outcome (e.g., being discharged from the hospital, being administered a treatment such as medication, undergoing a clinical procedure such as surgical operation, etc.). Such a period of time may be, for example, about 1 hour, about 2 hours, about 3 hours, about 4 hours, about 6 hours, about 8 hours, about 10 hours, about 12 hours, about 14 hours, about 16 hours, about 18 hours, about 20 hours, about 22 hours, about 24 hours, about 2 days, about 3 days, about 4 days, about 5 days, about 6 days, about 7 days, about 10 days, about 2 weeks, about 3 weeks, about 4 weeks, about 1 month, about 2 months, about 3 months, about 4 months, about 6 months, about 8 months, about 10 months, about 1 year, or more than about 1 year.

Input data and/or features provided to one or more predictive models and/or one or more machine learning algorithms may be structured by aggregating the data into bins or alternatively using a one-hot encoding. Input data and/or features may comprise feature values or vectors derived from the previously mentioned inputs, such as cross-correlations calculated between separate molecular signatures and/or features of the molecular signatures or other measurements over a fixed period of time, and the discrete derivative or the finite difference between successive measurements. Such a period of time may be, for example, about 1 hour, about 2 hours, about 3 hours, about 4 hours, about 6 hours, about 8 hours, about 10 hours, about 12 hours, about 14 hours, about 16 hours, about 18 hours, about 20 hours, about 22 hours, about 24 hours, about 2 days, about 3 days, about 4 days, about 5 days, about 6 days, about 7 days, about 10 days, about 2 weeks, about 3 weeks, about 4 weeks, about 1 month, about 2 months, about 3 months, about 4 months, about 6 months, about 8 months, about 10 months, about 1 year, or more than about 1 year.

Training records may be constructed from sequences of observations. Such sequences may comprise a fixed length for ease of data processing. For example, sequences may be zero-padded or selected as independent subsets of a single subject's records.

The one or more predictive models and/or one or more machine learning algorithms may process one or more input features to generate one or more output values comprising one or more phenotypes. For example, such phenotype may comprise a binary classification of a healthy/normal health state (e.g., absence of a disease or disorder) or an adverse health state (e.g., presence of a disease or disorder), a classification between a group of categorical labels (e.g., ‘no disease or disorder’, ‘apparent disease or disorder’, and ‘likely disease or disorder’), a likelihood (e.g., relative likelihood or probability) of developing a particular disease or disorder, a score indicative of a presence of disease or disorder, a score indicative of a level of systemic inflammation experienced by the patient, a ‘risk factor’ for the likelihood of mortality of the patient, a prediction of the time at which the patient is expected to have developed the disease or disorder, a confidence interval for any numeric predictions, or any combination thereof. Various predictive model and/or machine learning algorithms may be cascaded such that the output of one or more predictive models and/or one or more machine learning algorithms may be used as one or more input features to subsequent layers or subsections of the one or more predictive model and/or one or more machine learning algorithms.

In order to train the one or more predictive models and/or the one or more machine learning algorithms (e.g., by determining weights and correlations of the predictive model and/or the machine learning algorithm) to generate real-time classifications or predictions, the model can be trained using datasets (e.g., training datasets), described elsewhere herein. Such datasets may be sufficiently large to generate statistically significant classifications or predictions. For example, datasets may comprise databases of de-identified data including one or more molecular signatures, other measurements from a hospital or other clinical setting, or any combination thereof.

Datasets, as described elsewhere herein, may be split into subsets (e.g., discrete or overlapping), such as a training dataset, a development dataset, and a test dataset. For example, a dataset may be split into a training dataset comprising 80% of the dataset, a development dataset comprising 10% of the dataset, and a test dataset comprising 10% of the dataset. The training dataset may comprise about 10%, about 20%, about 30%, about 40%, about 50%, about 60%, about 70%, about 80%, or about 90% of the dataset. The development dataset may comprise about 10%, about 20%, about 30%, about 40%, about 50%, about 60%, about 70%, about 80%, or about 90% of the dataset. The test dataset may comprise about 10%, about 20%, about 30%, about 40%, about 50%, about 60%, about 70%, about 80%, or about 90% of the dataset. Training sets (e.g., training datasets) may be selected by random sampling of a set of data corresponding to one or more subject cohorts to ensure independence of sampling. In some cases, training sets (e.g., training datasets) may be selected by proportionate sampling of a set of data corresponding to one or more subject cohorts to ensure independence of sampling.

To improve the accuracy of predictive model and/or machine learning algorithm predictions and reduce overfitting of the predictive model and/or machine learning algorithm, the datasets may be augmented to increase the number of samples within the training set. For example, data augmentation may comprise rearranging the order of observations in a training record. To accommodate datasets having missing observations, methods to impute missing data may be used, such as forward-filling, back-filling, linear interpolation, and multi-task Gaussian processes. Datasets may be filtered to remove confounding factors. For example, within a database, a subset of subjects may be excluded.

Neural network techniques, such as dropout or regularization, may be used during training the one or more predictive models and/or one or more machine learning algorithms to prevent overfitting. The neural network may comprise a plurality of sub-networks, each of which is configured to generate a classification or prediction of a different type of output information (e.g., which may be combined to form an overall output of the neural network). The one or more predictive models and/or the one or more machine learning algorithms may alternatively utilize statistical or related algorithms including random forest, classification and regression trees, support vector machines, discriminant analyses, regression techniques, ensemble and gradient-boosted variations thereof, or any combination thereof.

When the one or more predictive models and/or the one or more machine learning algorithms generate a classification or a prediction of a phenotype, a notification (e.g., alert or alarm) may be generated and transmitted to a health care provider, such as a physician, nurse, health care personnel managing, or any combination thereof, treating a subject e.g., a subject within a hospital. Notifications may be transmitted via an automated phone call, a short message service (SMS), multimedia message service (MMS) message, an e-mail, an alert within a dashboard, or any combination thereof. The notification may comprise output information such as a prediction of a phenotype, a likelihood of the phenotype, a time until an expected onset of the phenotype, a confidence interval of the likelihood or time, a recommended course of treatment for a phenotype, or any combination thereof.

To validate the performance of the one or more predictive models and/or one more machine learning algorithms, different performance metrics may be generated. For example, an area under the receiver-operating curve (AUROC) may be used to determine the diagnostic and/or classification capability of the one or more predictive models and/or one or more machine learning algorithms. For example, the one or more predictive models and/or one or more machine learning algorithms may use classification thresholds which are adjustable, such that specificity and sensitivity are tunable, and the receiver-operating characteristic curve (ROC) can be used to identify the different operating points corresponding to different values of specificity and sensitivity of the one or more predictive models and/or one or more machine learning algorithms.

In some cases, such as when datasets are not sufficiently large, cross-validation may be performed to assess the robustness of one or more predictive models and/or one or more machine learning algorithms across different training and testing datasets.

To calculate performance metrics such as sensitivity, specificity, accuracy, positive predictive value (PPV), negative predictive value (NPV), area under the precision recall curve (AUPRC_, area under the receiver operating characteristic curve (AUROC), any combination thereof, or similar, the following definitions may be used. A “false positive” may refer to an outcome in which a positive outcome or result has been incorrectly or prematurely generated (e.g., before the actual onset of, or without any onset of a phenotype). A “true positive” may refer to an outcome in which positive outcome or result has been correctly generated, when the subject has the phenotype (e.g., the subject shows symptoms of the phenotype, or the subject's record indicates the phenotype). A “false negative” may refer to an outcome in which a negative outcome or result has been generated, but the subject has the phenotype (e.g., the subject shows symptoms of the phenotype, or the subject's record indicates the phenotype). A “true negative” may refer to an outcome in which a negative outcome or result has been generated (e.g., before the actual onset of, or without any onset of, the phenotype).

The one or more predictive models and/or one or more machine learning algorithms may be trained until certain pre-determined conditions for accuracy or performance are satisfied, such as having minimum desired values corresponding to classification and/or diagnostic accuracy measures. For example, the diagnostic accuracy measure may correspond to prediction of a likelihood of occurrence of a phenotype of the subject. As another example, the diagnostic accuracy measure may correspond to prediction of a likelihood of deterioration or recurrence of a phenotype for which the subject has previously been treated. Examples of diagnostic accuracy measures may include sensitivity, specificity, positive predictive value (PPV), negative predictive value (NPV), accuracy, area under the precision-recall curve (AUPRC), and area under the curve (AUC) of a Receiver Operating Characteristic (ROC) curve (AUROC) corresponding to the diagnostic accuracy of detecting or predicting a phenotype.

For example, such a pre-determined condition may be that the sensitivity of predicting the phenotype comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the specificity of predicting the phenotype a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the positive predictive value (PPV) of predicting the phenotype comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the negative predictive value (NPV) of predicting the phenotype comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the area under the curve (AUC) of a Receiver Operating Characteristic (ROC) curve (AUROC) of predicting the phenotype comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

As another example, such a pre-determined condition may be that the area under the precision-recall curve (AUPRC) of predicting the phenotype comprises a value of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the phenotype with a sensitivity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the phenotype with a specificity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the phenotype with a positive predictive value (PPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the phenotype with a negative predictive value (NPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the phenotype with an area under the curve (AUC) of a Receiver Operating Characteristic (ROC) curve (AUROC) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the phenotype with an area under the precision-recall curve (AUPRC) of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

The training data sets may be collected from training subjects (e.g., humans). Each training subject has a diagnostic status indicating that they have either been diagnosed and/or classified with the phenotype or have not been diagnosed with the phenotype. In some cases, the training subject comprises one or more humans. In some embodiments, the training subjects may be children aged equal to, or below, 12 years (e.g., equal to or below 5 years, 4 years, 3 years, 2 years, 1 year, 9 months, 6 months, 3 months or 1 month). In some embodiments, the child may be between the ages of about 12 and about 5 years old. In some embodiments, the subject may be less than about 12, 11, 10, 9, 8, 7, 5, 4, 3, 2, or 1 year(s) old. In some embodiments, the subject may be at least about 1, 2, 3, 4, 5, 6, 7, 8, 9, 10, 11, or 12 year(s) old. The training procedure, as described elsewhere herein may be performed for each training subject in a plurality of training subjects.

In some embodiments, the one or more predictive models and/or the one or more machine learning algorithms may comprise a neural network or a convolutional neural network. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

Independent component analysis (ICA), described elsewhere herein, in the unsupervised dimensionality-reduction of molecular signatures, is described in Lee, T.-W. (1998): Independent component analysis: Theory and applications, Boston, Mass: Kluwer Academic Publishers, ISBN 0-7923-8261-7, and Hyvärinen, A.; Karhunen, J.; Oja, E. (2001): Independent Component Analysis, New York: Wiley, ISBN 978-0-471-40540-5, which is hereby incorporated by reference in its entirety.

Principal component analysis (PCA), described elsewhere herein, in the unsupervised dimensionality-reduction of molecular signatures, is described in Jolliffe, I. T. (2002). Principal Component Analysis. Springer Series in Statistics. New York: Springer-Verlag. doi:10.1007/b98835. ISBN 978-0-387-95442-4, which is hereby incorporated by reference in its entirety.

SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs may separate a given set of binary labeled data with a hyper-plane that is maximally distant from the labeled data. For cases in which no linear separation is possible, SVMs can work in combination with the technique of ‘kernels’, which may automatically realize a non-linear mapping to a feature space. The hyper-plane found by the SVM in feature space may corresponds to a non-linear decision boundary in the input space.

Decision trees are described generally by Duda, 2001, Pattern Classification, John Wiley & Sons, Inc., New York, pp. 395-396, which is hereby incorporated by reference. Tree-based methods may partition the feature space into a set of rectangles, and then fit a model (like a constant) in each one. In some embodiments, the decision tree may be a random forest regression. One specific algorithm that can be used is a classification and regression tree (CART). Other specific decision tree algorithms may include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

Clustering (e.g., unsupervised clustering model algorithms and supervised clustering model algorithms) is described e.g., at pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem may be described as one of finding natural groupings in a dataset. To identify natural groupings, two issues may be addressed. First, a way to measure similarity (or dissimilarity) between two samples is determined. This metric (similarity measure) may be used to ensure that the samples in one cluster are more like one another than they are to samples in other clusters. Second, a mechanism for partitioning the data into clusters using the similarity measure may be determined. Similarity measures are discussed e.g., in Section 6.7 of Duda 1973, where it is stated that one way to begin a clustering investigation may be to define a distance function and to compute the matrix of distances between all pairs of samples in the training set. If distance is a good measure of similarity, then the distance between reference entities in the same cluster may be significantly less than the distance between the reference entities in different clusters. However, as stated on page 215 of Duda 1973, clustering may not require the use of a distance metric. For example, a nonmetric similarity function s(x, x′) can be used to compare two vectors x and x′. Conventionally, s(x, x′) may be a symmetric function whose value may be large when x and x′ are somehow “similar.” An example of a nonmetric similarity function s(x, x′) is provided e.g., on page 218 of Duda 1973. Once a method for measuring “similarity” or “dissimilarity” between points in a dataset has been selected, clustering may require a criterion function that measures the clustering quality of any partition of the data. Partitions of the data set that extremize the criterion function may be used to cluster the data. See e.g., page 217 of Duda 1973. Criterion functions are discussed e.g., in Section 6.8 of Duda 1973. More recently, Duda et al., Pattern Classification, 2^{nd} edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, New Jersey, each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, Jarvis-Patrick clustering, or any combination thereof. In some embodiments, the clustering may comprises unsupervised clustering, where no preconceived notion of what clusters should form when the training set is clustered, are imposed.

Regression models, such as that of the multi-category logit models, are described in e.g., Agresti, An Introduction to Categorical Data Analysis, 1996, John Wiley & Sons, Inc., New York, Chapter 8, which is hereby incorporated by reference in its entirety. In some embodiments, the one or more predictive model and/or one or more machine learning algorithms may make use of a regression model disclosed in Hastie et al., 2001, The Elements of Statistical Learning, Springer-Verlag, New York, which is hereby incorporated by reference in its entirety. In some embodiments, gradient-boosting models may be used toward, for example, the classification algorithms described herein; these gradient-boosting models are described in Boehmke, Bradley: Greenwell. Brandon (2019). “Gradient Boosting”. Hands-On Machine Learning with R. Chapman & Hall. pp. 221-245. ISBN 978-1-138-49568-5, which is hereby incorporated by reference in its entirety. In some embodiments, ensemble modeling techniques may be used, for example, toward the classification algorithms described herein; these ensemble modeling techniques are described in the implementation of classification models herein, are described in Zhou Zhihua (2012). Ensemble Methods: Foundations and Algorithms. Chapman and Hall/CRC. ISBN 978-1-439-83003-1, which is hereby incorporated by reference in its entirety.

In some embodiments, the machine learning analysis may be performed by a device executing one or more programs (e.g., one or more programs stored in the Non-Persistent Memory or in the Persistent Memory) including instructions to perform the data analysis. In some embodiments, the data analysis may be performed by a system comprising at least one processor (e.g., the processing core) and memory (e.g., one or more programs stored in the Non-Persistent Memory or in the Persistent Memory) comprising instructions to perform the data analysis.

### EXAMPLES

The following examples are included for illustrative purposes only and are not intended to limit the scope of the invention.

### Example 1: Concentration of Organic Molecule Signatures and Association with Autism Spectrum Disorder

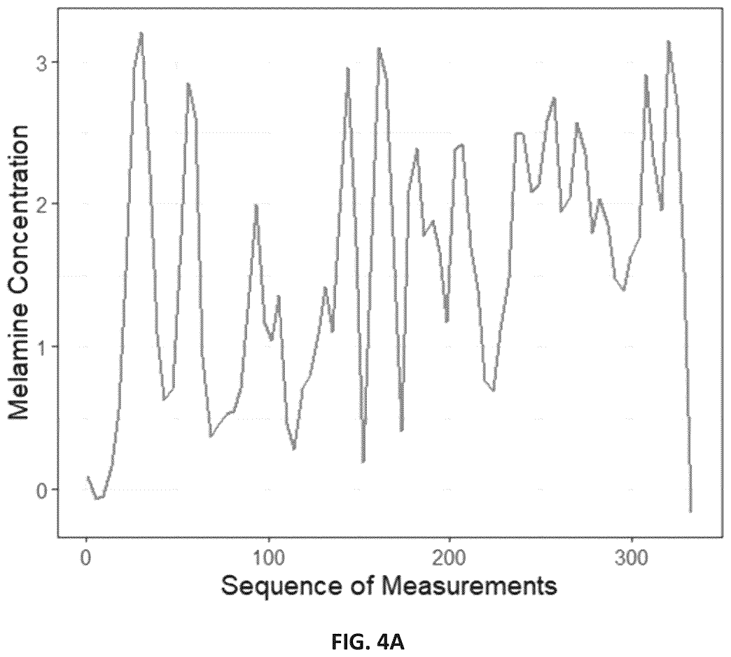

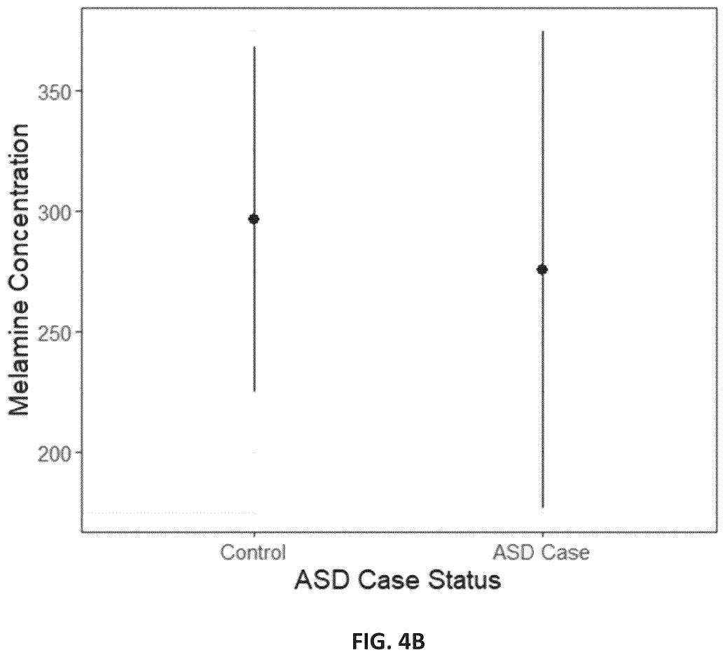

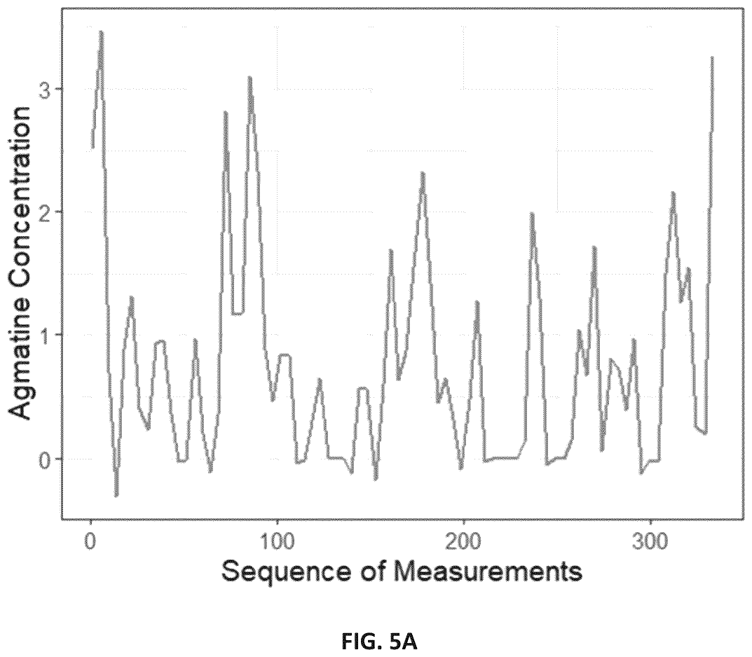

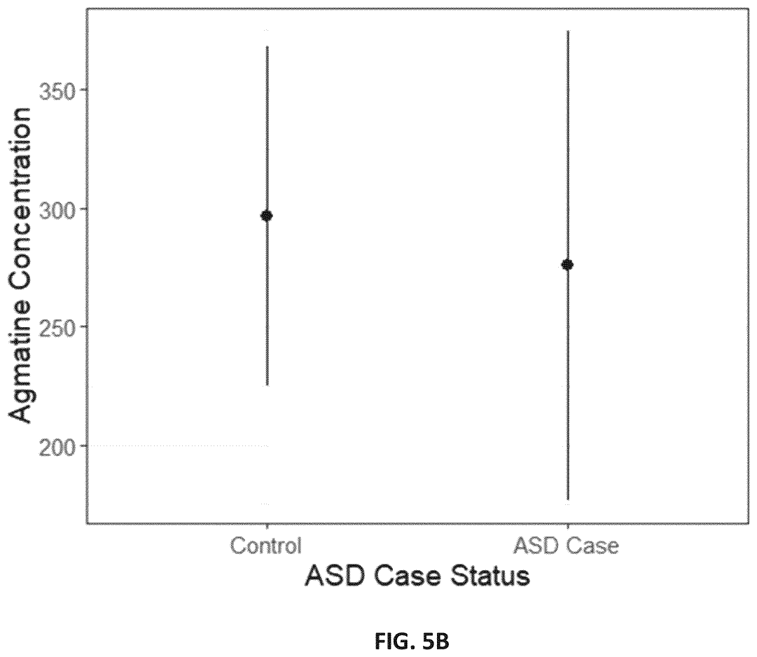

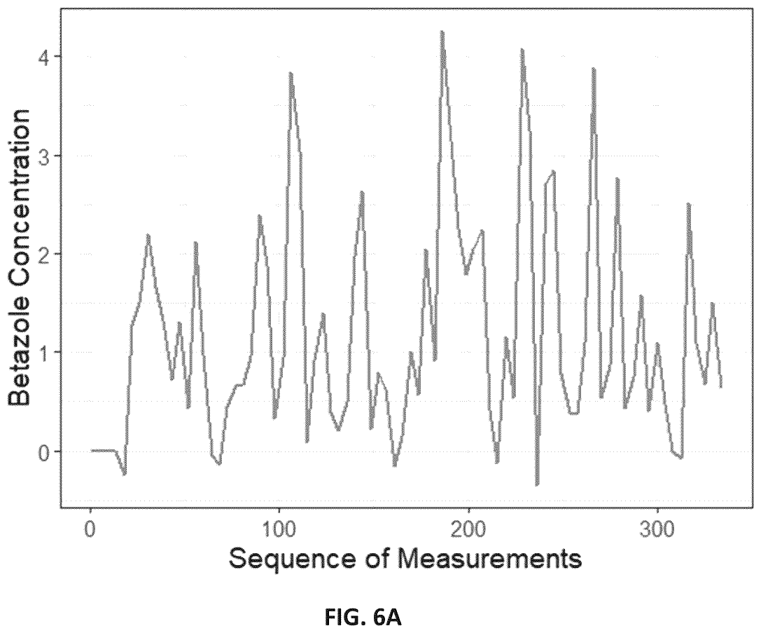

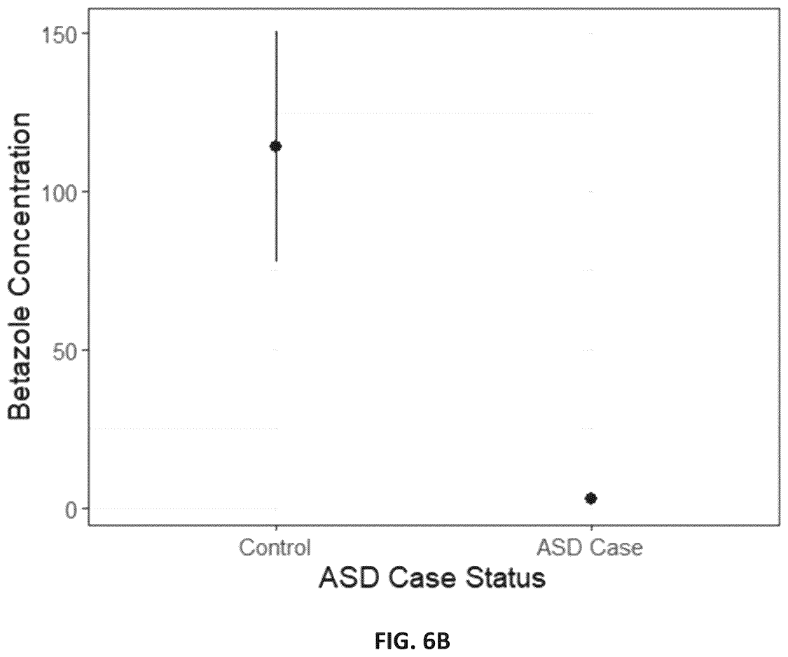

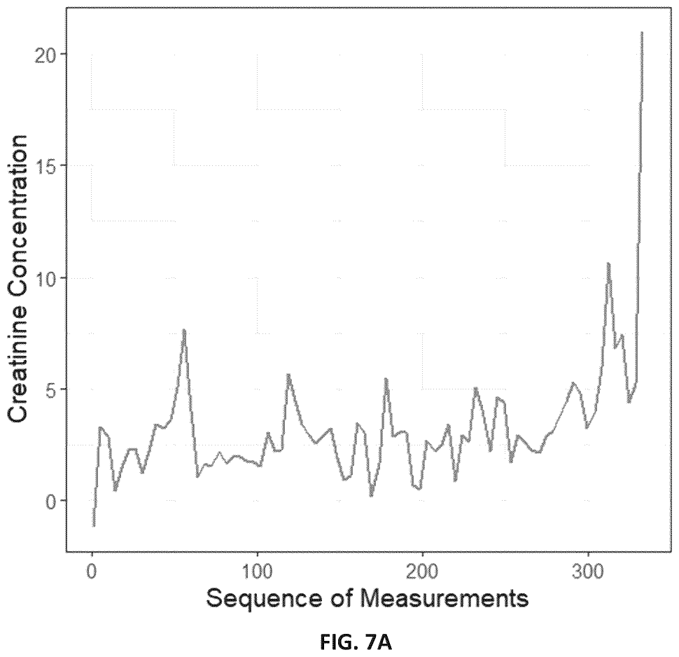

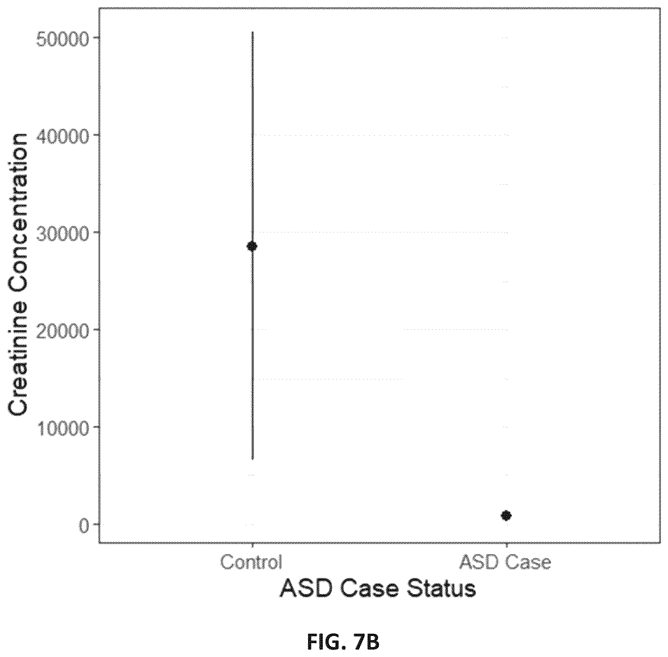

As described by the methods and/or systems provided herein, organic molecule signatures were obtained from a set of subjects' hair biological samples to determine whether the average concentration of the organic molecule signatures differentiate subjects belonging to control and autism spectrum disorder groups. Namely, the organic molecule signatures analyzed included: hypotaurine (FIG. 3A), melamine (FIG. 4A), agmatine (FIG. 5A), Betazole (FIG. 6A), and creatine (FIG. 7A), as shown by representative organic molecule signature concentration measurements. The geometric mean of all concentration values for each organic molecule signature across all subjects between the two groups was determined and plotted with standard error of the mean for each molecule (FIGS. 3B, 4B, 5B, 6B, and 7B). From these results, it can be seen that the geometric mean of the concentration for each organic molecule signature provides a feature that distinguishes the two groups.

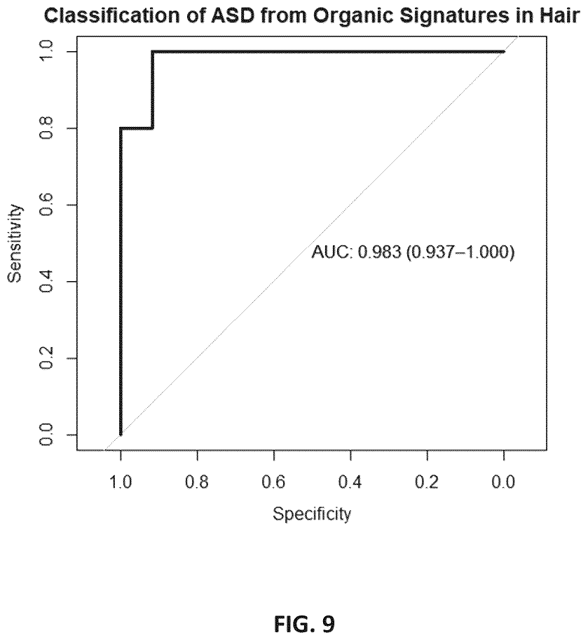

The concentration features of the organic molecule signatures in combination with a label of subject group designation (i.e., healthy subjects or diseased subjects with autism spectrum disorder) were used to train a predictive model. The trained predictive model was then used on a subject's sample that was not included in the training data to determine the presence of autism spectrum disorder. The performance of the predictive model can be seen from the receiver operating characteristic (ROC) curve and area under the ROC curve, as shown in FIG. 9. The predictive model performed classifications of autism spectrum disorder phenotype for subjects with a sensitivity of 80% and specificity of 100%. The area under the curve for the predictive model was 0.983.

### Example 2: Organic Molecule Exposure Signatures

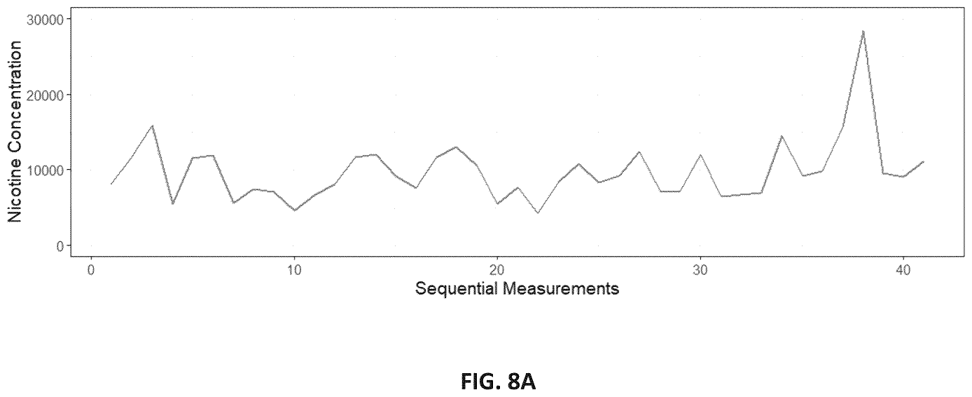

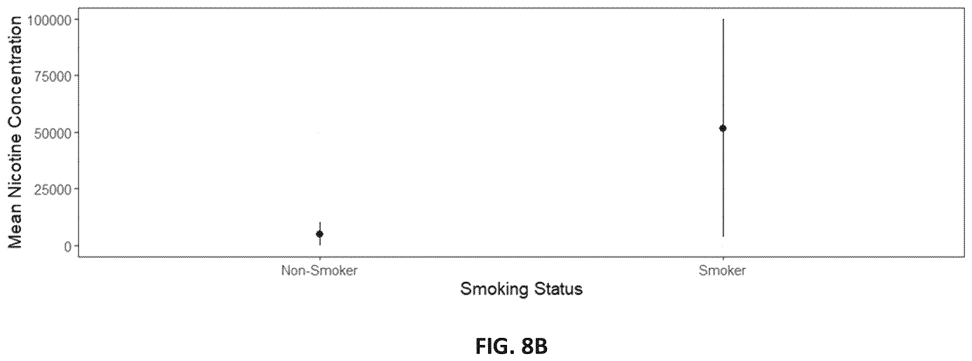

Using the methods and/or systems of the disclosure, described elsewhere herein, the concentration of nicotine organic molecule signatures of a group of smokers and non-smokers hair biological samples was measured to determine if nicotine organic molecule signatures classify and/or distinguish smokers and non-smokers, as seen in FIGS. 8A-8B. from FIGS. 8A-8B, it can be seen that average concentration of nicotine organic molecule signatures (FIG. 8B) does differentiate groups of smokers and non-smokers.

### Example 3: Probe Based Organic Molecule Signatures

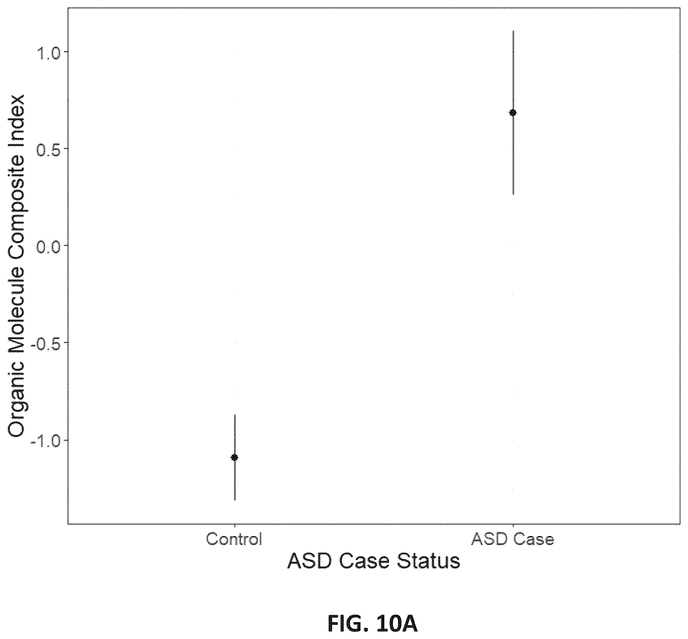

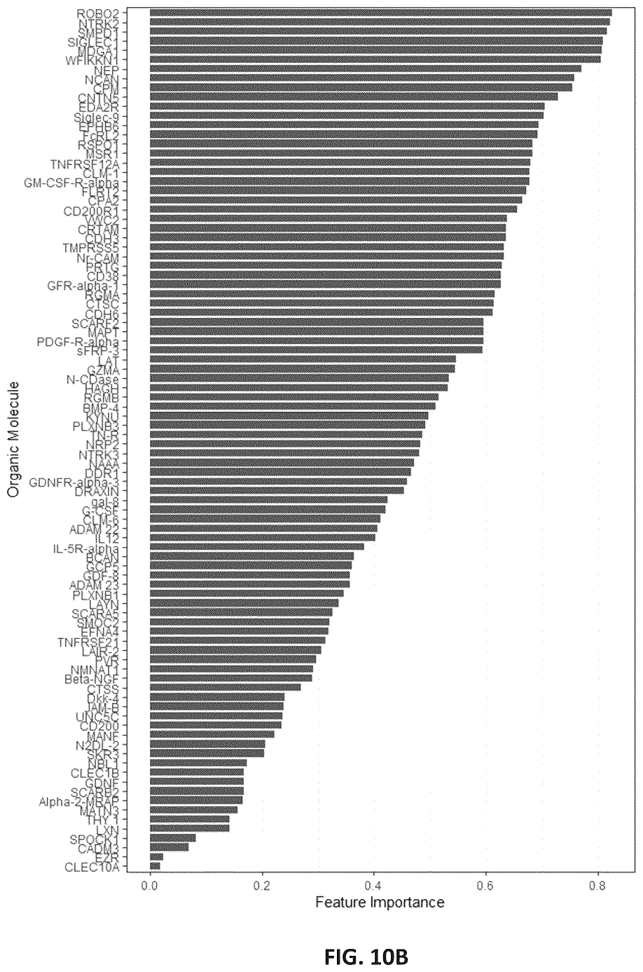

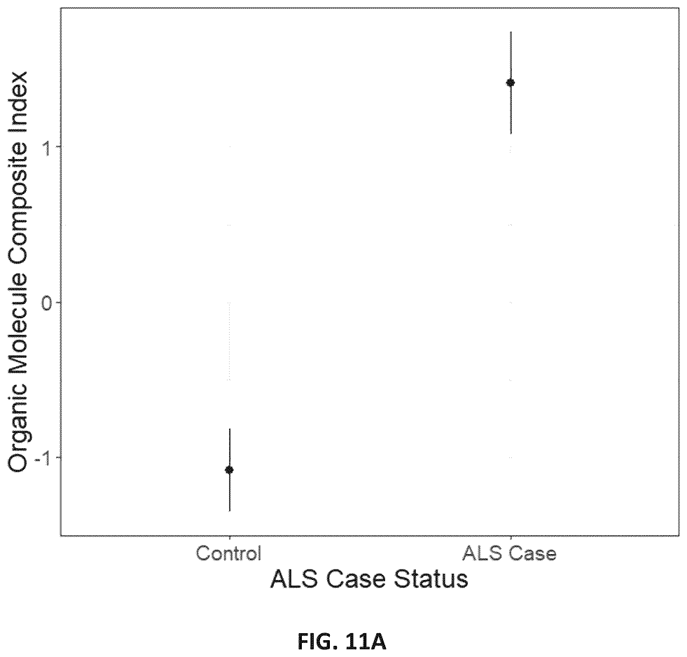

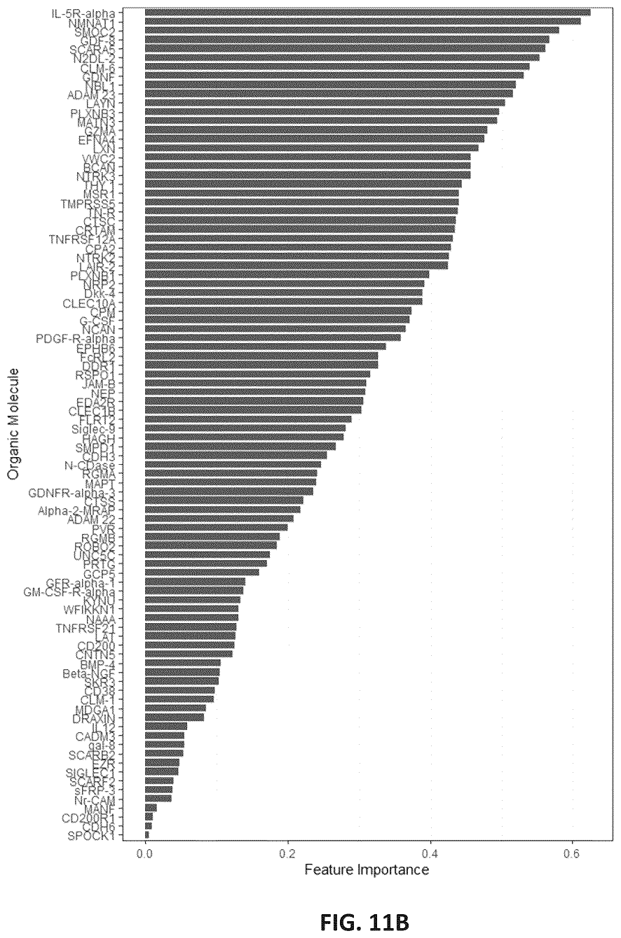

Using the methods and/or systems of the disclosure, described elsewhere herein, one or more organic molecule signatures of control, disease autism spectrum disorder (FIGS. 10A-10B) phenotype, and disease amyotrophic lateral sclerosis (FIGS. 11A-11B) phenotype, were determined by disposing one or more probes, described elsewhere herein, on teeth of subjects of the associated study groups. As described elsewhere herein, the one or more probes may target one or more proteins of the biological sample. The one or more probes, as described elsewhere herein, may comprise a targeted binding moiety that targets an organic molecule of a plurality of organic molecules. The resulting organic molecule signature of 100 organic compounds and their associated feature importance for autism spectrum disorder and amyotrophic lateral sclerosis are shown in FIG. 10B and FIG. 11B, respectively. A composite index, determined by linear discriminant analysis, was determined for each group of organic molecule signatures, and was plotted against control and disease groups, ASD shown on FIG. 10A, and ALS shown on FIG. 11A, with error bars indicating the standard error of the mean for the composite index. From FIGS. 10A, and 11A it can be seen that the composite index score for the organic molecules identified differentiate the control and disease groups.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following embodiments and claims define the scope of the invention and that methods and structures within the scope of these claims and their equivalents be covered thereby.

### EMBODIMENTS

Embodiment 1 comprises a method for classifying a phenotype of a subject, comprising: obtaining or collecting one or more organic molecule signatures from a plurality of positions along a biological sample of a subject; and determining the phenotype of the subject from the one or more organic molecule signatures of the subject.
Embodiment 2 comprises the method of embodiment 1, wherein the one or more organic molecule signatures comprise a molecular signature of a molecule with a mass to charge ratio of at least 50 Daltons (Da).
Embodiment 3 comprises the method of embodiments 1 or 2, wherein the one or more organic molecule signatures comprise one or more time-resolved organic molecule signatures.
Embodiment 4 comprises the method of any one of embodiments 1-3, wherein a light source is used to obtain or collect the one or more organic molecule signatures from the plurality of positions along the biological sample.
Embodiment 5 comprises the method of embodiment 4, wherein the light source comprises a laser, pulsed laser, continuous wave laser, or any combination thereof.
Embodiment 6 comprises the method of any one of embodiments 1-5, wherein the phenotype of the subject comprises molecular phenotype, physiologic phenotype, a behavioral phenotype, a disease phenotype, a healthy phenotype, an exposure phenotype, one or more upregulated or downregulated physiological pathways, pharmaceutical response, nutraceutical response, presence of exogenous compounds, presence of endogenous compounds, presence of inflammation, or any combination thereof.
Embodiment 7 comprises the method of embodiment 6, wherein the disease phenotype comprises autism spectrum disorder (ASD), attention-deficit hyperactivity disorder, amyotrophic lateral sclerosis, or any combination thereof.
Embodiment 8 comprises the method of embodiments 6 or 7, wherein the exogenous compounds comprise nicotine, melamine, or any combination thereof.
Embodiment 9 comprises the method of any one of embodiments 6-8, wherein the endogenous compounds comprise endogenous metabolites, signaling molecules, or any combination thereof.
Embodiment 10 comprises the method of embodiment 9, wherein the signaling molecules comprise hypotaurine.
Embodiment 11 comprises the method of embodiments 9 or 10, wherein the endogenous metabolites comprise creatinine.
Embodiment 12 comprises the method of any one of embodiments 6-11, wherein the nutraceutical comprises agmatine.
Embodiment 13 comprises the method of any one of embodiments 6-12, wherein the pharmaceutical comprises a pharmaceutical to treat heart burn, acid reflux, peptic ulcers, or any combination thereof.
Embodiment 14 comprises the method of embodiment 13, wherein the pharmaceutical comprises Betazole.
Embodiment 15 comprises the method of any one of embodiments 6-14, wherein the exposure phenotype comprises exposure of the subject to environmental chemicals.
Embodiment 16 comprises the method of any one of embodiments 1-15, wherein the biological sample comprises hair, teeth, fingernails, toenails, or any combination thereof samples.
Embodiment 17 comprises the method of any one of embodiments 1-16, wherein obtaining or collecting comprises conducting matrix assisted laser desorption ionization-time of flight mass spectrometry (MALDI-ToF MS), laser ablation electrospray ionization (LAESI), protein fluorescence assays, or any combination thereof with the biological sample.
Embodiment 18 comprises the method of any one of embodiments 1-17, wherein the subject has been administered or is taking a pharmaceutical, nutraceutical, or a combination thereof to treat a condition of the subject, and wherein the phenotype of the subject comprises a response to the pharmaceutical, nutraceutical, or a combination thereof.
Embodiment 19 comprises the method of embodiment 18, wherein the phenotype is used to determine an adjustment of the pharmaceutical, nutraceutical, or a combination thereof, to ameliorate the condition of the subject.
Embodiment 20 comprises the method of embodiment 19, wherein the adjustment of the pharmaceutical, nutraceutical, or a combination thereof comprises administering modified or new pharmaceutical, nutraceutical, or a combination thereof.
Embodiment 21 comprises the method of any one of embodiments 1-20, wherein the one or more organic molecule signatures comprise temporal concentrations of one or more organic molecules obtained or collected from the biological sample of the subject.
Embodiment 22 comprises the method of any one of embodiments 1-21, wherein determining the phenotype of the subject comprises: training a predictive model with one or more organic molecule signatures and associated phenotype labels of a set of subjects different than the subject; and providing the subject's one or more organic molecule signatures to the trained predictive model and outputting the phenotype of the subject.
Embodiment 23 comprises a method for classifying a phenotype of a subject, comprising: obtaining or collecting one or more molecular signatures of molecules with a mass to charge ratio of at least 50 Da from a biological sample of a subject when one or more probes are disposed on the biological sample; and determining the phenotype of the subject from the one or more molecular signatures of the subject.
Embodiment 24 comprises the method of embodiment 23, wherein the one or more molecular signatures comprise one or more organic molecule signatures.
Embodiment 25 comprises the method of embodiments 23 or 24, wherein a light source is used to obtain or collect the one or more molecular signatures from one or more probes disposed on the biological sample.
Embodiment 26 comprises the method of embodiment 25, wherein the light source comprises a laser, pulsed laser, continuous wave laser, or any combination thereof.
Embodiment 27 comprises the method of any one of embodiments 23-26, wherein the phenotype of the subject comprises a molecular phenotype, physiologic phenotype, behavioral phenotype, a disease phenotype, a healthy phenotype, an exposure phenotype, one or more upregulated or downregulated physiological pathways, pharmaceutical response, nutraceutical response, presence of exogenous compounds, presence of endogenous compounds, presence of inflammation, or any combination thereof.
Embodiment 28 comprises the method of embodiment 27, wherein the disease phenotype comprises autism spectrum disorder (ASD), attention-deficit hyperactivity disorder, amyotrophic lateral sclerosis, or any combination thereof.
Embodiment 29 comprises the method of embodiments 27 or 28, wherein the exogenous compounds comprise nicotine, melamine, or any combination thereof.
Embodiment 30 comprises the method of any one of embodiments 27-29, wherein the endogenous compounds comprise endogenous metabolites, signaling molecules, or any combination thereof.
Embodiment 31 comprises the method of embodiment 30, wherein the signaling molecules comprise hypotaurine.
Embodiment 32 comprises the method of embodiments 30 or 31, wherein the endogenous metabolites comprise creatinine.
Embodiment 33 comprises the method of any one of embodiments 27-32, wherein the nutraceutical comprises agmatine.
Embodiment 34 comprises the method of any one of embodiments 27-33, wherein the pharmaceutical comprises a pharmaceutical to treat heart burnt, acid reflux, peptic ulcers, or any combination thereof.
Embodiment 35 comprises the method of embodiment 34, wherein the pharmaceutical comprises Betazole.
Embodiment 36 comprises the method of any one of embodiments 27-35, wherein the exposure phenotype comprises exposure of the subject to environmental chemicals.
Embodiment 37 comprises the method of any one of embodiments 23-36, wherein the biological sample comprises hair, teeth, fingernails, toenails, or any combination thereof samples.
Embodiment 38 comprises the method of any one of embodiments 23-37, wherein obtaining or collecting comprise conducting a protein fluorescence assay with one or more probes.
Embodiment 39 comprises the method of any one of embodiments 23-38, wherein the subject has been administered or is taking a pharmaceutical, nutraceutical, or a combination thereof to treat a condition of the subject, and wherein the phenotype of the subject comprises a response to the pharmaceutical, nutraceutical, or a combination thereof.
Embodiment 40 comprises the method of claim 39, wherein the phenotype is used to determine an adjustment of the pharmaceutical, nutraceutical, or a combination thereof, to ameliorate the condition of the subject.
Embodiment 41 comprises the method of embodiments 39 or 40, wherein the adjustment of the pharmaceutical, nutraceutical, or a combination thereof comprises administering modified or new pharmaceutical, nutraceutical, or a combination thereof.
Embodiment 42 comprises the method of any one of embodiments 23-41, wherein determining the phenotype of the subject comprises: training a predictive model with one or more molecular signatures and associated phenotype labels of a set of subjects different than the subject; and providing the subject's one or more molecular signatures to the trained predictive model and outputting the phenotype of the subject.
Embodiment 43 comprises a system for classifying a phenotype of a subject, comprising: one or more processor and memory storing one or more programs for execution by the one or more processors, the one or more programs comprising instructions to: (i) obtain or collect one or more organic molecule signatures of a biological sample of the subject from a plurality of positions along the biological sample; and (ii) determine the phenotype of the subject from the one or more organic molecule signatures of the subject.
Embodiment 44 comprises the system of embodiment 43, wherein the one or more organic molecule signatures comprise a molecular signature of a molecule with mass to charge ratio of at least 50 Daltons (Da).
Embodiment 45 comprises the system of embodiments 43 or 44, wherein the one or more organic molecule signatures comprise one or more time-resolved organic molecule signatures.
Embodiment 46 comprises the system of any one of embodiments 43-45, wherein a light source is used to obtain or collect the one or more organic molecule signatures from the plurality of positions along the biological sample.
Embodiment 47 comprises the system of embodiment 46, wherein the light source comprises a laser, pulsed laser, continuous wave laser, or any combination thereof.
Embodiment 48 comprises the system of any one of embodiments 43-47, wherein the phenotype of the subject comprises a molecular phenotype, physiologic phenotype, behavioral phenotype, a disease phenotype, a healthy phenotype, an exposure phenotype, one or more upregulated or downregulated physiological pathways, pharmaceutical response, nutraceutical response, presence of exogenous compounds, presence of endogenous compounds, presence of inflammation, or any combination thereof.
Embodiment 49 comprises the system of embodiment 48, wherein the disease phenotype comprises autism spectrum disorder (ASD), attention-deficit hyperactivity disorder, amyotrophic lateral sclerosis, or any combination thereof.
Embodiment 50 comprises the system of embodiments 48 or 49, wherein the exogenous compounds comprise nicotine, melamine, or any combination thereof.
Embodiment 51 comprises the system of any one of embodiments 48-50, wherein the endogenous compounds comprise endogenous metabolites, signaling molecules, or any combination thereof.
Embodiment 52 comprises the system of embodiment 51, wherein the signaling molecules comprise hypotaurine.
Embodiment 53 comprises the system of embodiments 51 or 52, wherein the endogenous metabolites comprise creatinine.
Embodiment 54 comprises the system of any one of embodiments 48-53, wherein the nutraceutical comprises agmatine.
Embodiment 55 comprises the system of any one of embodiments 48-54, wherein the pharmaceutical comprises a pharmaceutical to treat heart burnt, acid reflux, peptic ulcers, or any combination thereof.
Embodiment 56 comprises the system of embodiment 55, wherein the pharmaceutical comprises Betazole.
Embodiment 57 comprises the system of any one of embodiments 48-56, wherein the exposure phenotype comprises exposure of the subject to environmental chemicals.
Embodiment 58 comprises the system of any one of embodiments 43-57, wherein the biological sample comprises hair, teeth, fingernails, toenails, or any combination thereof samples.
Embodiment 59 comprises the system of any one of embodiments 43-58, wherein obtaining or collecting comprises conducting matrix assisted laser desorption ionization-time of flight mass spectrometry (MALDI-ToF MS), laser ablation electrospray ionization (LAESI), protein fluorescence assays, or any combination thereof with the biological sample.
Embodiment 60 comprises the system of any one of embodiments 43-59, wherein the subject has been administered or is taking a pharmaceutical, nutraceutical, or a combination thereof to treat a condition of the subject, and wherein the phenotype of the subject comprises a response to the pharmaceutical, nutraceutical, or a combination thereof.
Embodiment 61 comprises the system of embodiment 60, wherein the phenotype is used to determine an adjustment of the pharmaceutical, nutraceutical, or a combination thereof, to ameliorate the condition of the subject.
Embodiments 62 comprises the system of embodiment 61, wherein the adjustment of the pharmaceutical, nutraceutical, or a combination thereof comprises administering modified or new pharmaceutical, nutraceutical, or a combination thereof.
Embodiment 63 comprises the system of any one of embodiments 43-62, wherein the one or more organic molecule signatures comprise temporal concentrations of one or more organic molecules obtained or collected from the biological sample of the subject.
Embodiment 64 comprises the system of any one of embodiments 43-63, wherein determining the phenotype of the subject comprises: (i) training a predictive model with one or more organic molecule signatures and associated phenotype labels of a set of subjects different than the subject; and (ii) providing the subject's one or more organic molecule signatures to the trained predictive model and outputting the phenotype of the subject.
Embodiment 65 comprises a system for classifying a phenotype of a subject, comprising: one or more processor and memory storing one or more programs for execution by the one or more processors, the one or more programs comprising instructions to: (i) obtain or collect one or more molecular signatures of molecules with a mass to charge ratio of at least 50 Da from a biological sample of a subject when one or more probes are disposed on the biological sample; and (ii) determine the phenotype of the subject from the one or more molecular signatures of the subject.
Embodiment 66 comprises the system of embodiment 65, wherein the one or more molecular signatures comprise one or more organic molecule signatures.
Embodiment 67 comprises the system of embodiments 65 or 66, wherein a light source is used to obtain or collect the one or more molecular signatures from the biological sample.
Embodiment 68 comprises the system of embodiment 67, wherein the light source comprises a laser, pulsed laser, continuous wave laser, or any combination thereof.
Embodiment 69 comprises the system of any one of embodiments 65-68, wherein the phenotype of the subject comprises a molecular phenotype, physiologic phenotype, behavioral phenotype, a disease phenotype, a healthy phenotype, an exposure phenotype, one or more upregulated or downregulated physiological pathways, pharmaceutical response, nutraceutical response, presence of exogenous compounds, presence of endogenous compounds, presence of inflammation, or any combination thereof.
Embodiment 70 comprises the system of embodiment 69, wherein the disease phenotype comprises autism spectrum disorder (ASD), attention-deficit hyperactivity disorder, amyotrophic lateral sclerosis, or any combination thereof.
Embodiment 71 comprises the system of embodiments 69 or 70, wherein the exogenous compounds comprise nicotine, melamine, or any combination thereof.
Embodiment 72 comprises the system of any one of embodiments 69-71, wherein the endogenous compounds comprise endogenous metabolites, signaling molecules, or any combination thereof.
Embodiment 73 comprises the system of embodiment 72, wherein the signaling molecules comprise hypotaurine.
Embodiment 74 comprises the system of embodiments 72 or 73, wherein the endogenous metabolites comprise creatinine.
Embodiment 75 comprises the system of any one of embodiments 69-74, wherein the nutraceutical comprises agmatine.
Embodiment 76 comprises the system of any one of embodiments 69-75, wherein the pharmaceutical comprises a pharmaceutical to treat heart bumt, acid reflux, peptic ulcers, or any combination thereof.
Embodiment 77 comprises the system of any one of embodiments 69-76, wherein the pharmaceutical comprises Betazole.
Embodiment 78 comprises the system of any one of embodiments 69-77, wherein the exposure phenotype comprises exposure of the subject to environmental chemicals.
Embodiment 79 comprises the system of any one of embodiments 65-78, wherein the biological sample comprises hair, teeth, fingernails, toenails, or any combination thereof samples.
Embodiment 80 comprises the system of any one of embodiments 65-79, wherein the instruction of obtain or collect comprise conducting a protein fluorescence assay with the one or more probes.
Embodiment 81 comprises the system of any one of embodiments 65-80, wherein the subject has been administered or is taking a pharmaceutical, nutraceutical, or a combination thereof to treat a condition of the subject, and wherein the phenotype of the subject comprises a response to the pharmaceutical, nutraceutical, or a combination thereof.
Embodiment 82 comprises the system of embodiment 81, wherein the phenotype is used to determine an adjustment of the pharmaceutical, nutraceutical, or a combination thereof, to ameliorate the condition of the subject.
Embodiment 83 comprises the system of embodiment 82, wherein the adjustment of the pharmaceutical, nutraceutical, or a combination thereof comprises administering modified or new pharmaceutical, nutraceutical, or a combination thereof.
Embodiment 84 comprises the system of any one of embodiments 65-83, wherein the instruction of determine the phenotype of the subject comprises: train a predictive model with one or more molecular signatures and associated phenotype labels of a set of subjects different than the subject; and provide the subject's one or more molecular signatures to the trained predictive model and output the phenotype of the subject.
Embodiment 85 comprises a method of training an untrained or partially untrained machine learning algorithm or predictive model, comprising: at a computer system having one or more processors, and memory storing one or more programs for execution by the one or more processors: collecting or obtaining one or more organic molecule signatures of a biological sample of a plurality of training subjects, wherein a first subset of training subjects in the plurality of training subjects have a first phenotype associated with the one or more organic molecule signatures and a second subset of training subjects in the plurality of training subjects have a second phenotype associated with the one or more organic molecule signatures; and training the untrained or partially untrained machine learning algorithm or predictive model with the one or more organic molecule signatures of the plurality of training subjects and the corresponding first phenotype and second phenotype associated with the one or more organic molecular signatures thereby producing or generating a trained predictive model.
Embodiment 86 comprises the method of embodiment 85, wherein the one or more organic molecule signatures comprise one or more organic molecule signatures of molecules with a mass to charge ratio of at least 50 Da.
Embodiment 87 comprises the method of embodiment 85 or 0, wherein the one or more organic molecule signatures comprise one or more time-resolved organic molecule signatures.
Embodiment 88 comprises the method of any one of embodiments 85-87, wherein a light source is used to obtain or collect the one or more organic molecule signatures from the biological sample of the plurality of training subjects.
Embodiment 89 comprises the method of embodiment 88, wherein the light source comprises a laser, pulsed laser, continuous wave laser, or any combination thereof.
Embodiment 90 comprises the method of any one of embodiments 85-89, wherein the first phenotype or the second phenotype comprise a molecular phenotype, physiologic phenotype, a behavioral phenotype, a disease phenotype, a healthy phenotype, an exposure phenotype, one or more upregulated or downregulated physiological pathways, pharmaceutical response, nutraceutical response, presence of exogenous compounds, presence of endogenous compounds, presence of inflammation, or any combination thereof.
Embodiment 91 comprises the method of embodiment 90, wherein the disease phenotype comprises autism spectrum disorder (ASD), attention-deficit hyperactivity disorder, amyotrophic lateral sclerosis, or any combination thereof.
Embodiment 92 comprises the method of embodiments 90 or 91, wherein the exogenous compounds comprise nicotine, melamine, or any combination thereof.
Embodiment 93 comprises the method of any one of embodiments 90-92, wherein the endogenous compounds comprise endogenous metabolites, signaling molecules, or any combination thereof.
Embodiment 94 comprises the method of embodiment 93, wherein the signaling molecules comprise hypotaurine.
Embodiment 95 comprises the method of embodiments 93 or 94, wherein the endogenous metabolites comprise creatinine.
Embodiment 96 comprises the method of any one of embodiments 90-95, wherein the nutraceutical comprises agmatine.
Embodiment 97 comprises the method of any one of embodiments 90-96, wherein the pharmaceutical comprises a pharmaceutical to treat heart burn, acid reflux, peptic ulcers, or any combination thereof.
Embodiment 98 comprises the method of any one of embodiments 90-97, wherein the pharmaceutical comprises Betazole.
Embodiment 99 comprises the method of any one of embodiments 90-98, wherein the exposure phenotype comprises exposure of the subject to environmental chemicals.
Embodiment 100 comprises the method of any one of embodiments 85-99, wherein the biological sample comprises hair, teeth, fingernails, toenails, or any combination thereof samples.
Embodiment 101 comprises the method of any one of embodiments 85-100, wherein obtaining or collecting comprises conducting matrix assisted laser desorption ionization-time of flight mass spectrometry (MALDI-ToF MS), laser ablation electrospray ionization (LAESI), protein fluorescence assays, or any combination thereof with the biological sample.
Embodiment 102 comprises the method of any one of embodiments 85-101, wherein the plurality of training subjects have been administered or is taking a pharmaceutical, nutraceutical, or a combination thereof to treat a condition of the plurality of training subjects, and wherein the first phenotype or the second phenotype of the plurality of training subjects comprises a response to the pharmaceutical, nutraceutical, or a combination thereof.
Embodiment 103 comprises the method of embodiment 102, wherein the first phenotype or the second phenotype is used to determine an adjustment of the pharmaceutical, nutraceutical, or a combination thereof, to ameliorate the condition of the subject.
Embodiment 104 comprises the method of embodiment 103, wherein the adjustment of the pharmaceutical, nutraceutical, or a combination thereof comprises administering modified or new pharmaceutical, nutraceutical, or a combination thereof.
Embodiment 105 comprises the method of any one of embodiments 85-104, wherein the one or more organic molecule signatures comprise temporal concentrations of the one or more organic molecules signatures obtained or collected from the biological sample of the plurality of training subjects.

1.-105. (canceled)

106. A method for classifying a phenotype of a subject, the method comprising:
obtaining or collecting one or more organic molecule signatures from a plurality of positions along a biological sample of a subject; and
determining the phenotype of the subject from the one or more organic molecule signatures.

107. The method of claim 106, wherein the one or more organic molecule signatures comprise a molecular signature of a molecule with a mass to charge ratio of at least 50 Daltons (Da).

108. The method of claim 106, wherein the one or more organic molecule signatures comprise one or more time-resolved organic molecule signatures.

109. The method of claim 106, wherein a light source is used to obtain or collect the one or more organic molecule signatures from the plurality of positions along the biological sample.

110. The method of claim 109, wherein the light source comprises a laser, pulsed laser, continuous wave laser, or any combination thereof.

111. The method of claim 106, wherein the phenotype of the subject comprises a molecular phenotype, a physiologic phenotype, a behavioral phenotype, a disease phenotype, a healthy phenotype, an exposure phenotype, one or more upregulated or downregulated physiological pathways, a pharmaceutical response, a nutraceutical response, a presence of exogenous compounds, a presence of endogenous compounds, a presence of inflammation, or any combination thereof.

112. The method of claim 111, wherein the disease phenotype comprises autism spectrum disorder (ASD), attention-deficit/hyperactivity disorder, amyotrophic lateral sclerosis, or any combination thereof.

113. The method of claim 111, wherein the exogenous compounds comprise nicotine, melamine, or any combination thereof.

114. The method of claim 111, wherein the endogenous compounds comprise endogenous metabolites, signaling molecules, or any combination thereof.

115. The method of claim 114, wherein the signaling molecules comprise hypotaurine.

116. The method of claim 114, wherein the endogenous metabolites comprise creatinine.

117. The method of claim 111, wherein a nutraceutical of the nutraceutical response comprises agmatine.

118. The method of claim 111, wherein a pharmaceutical of the pharmaceutical response comprises a pharmaceutical to treat heart burn, acid reflux, peptic ulcers, or any combination thereof.

119. The method of claim 118, wherein the pharmaceutical comprises Betazole.

120. The method of claim 111, wherein the exposure phenotype comprises exposure of the subject to environmental chemicals.

121. The method of claim 106, wherein the biological sample comprises hair, teeth, fingernails, toenails, or any combination thereof.

122. The method of claim 106, wherein determining the phenotype of the subject comprises:
training a predictive model with one or more organic molecule signatures and associated phenotype labels of a set of subjects different than the subject.

123. The method of claim 122, wherein the method further comprises providing the subject's one or more organic molecule signatures to the trained predictive model and outputting the phenotype of the subject.

124. A system for classifying a phenotype of a subject, the method comprising:
one or more processors and memory storing one or more programs for execution by the one or more processors, the one or more programs comprising instructions to:
(i) obtain or collect one or more molecular signatures of molecules with a mass to charge ratio of at least 50 Da from a biological sample of a subject when one or more probes are disposed on the biological sample; and
(ii) determine the phenotype of the subject from the one or more molecular signatures.

125. A method of training an untrained or partially untrained machine learning algorithm or predictive model, the method comprising:
at a computer system having one or more processors, and memory storing one or more programs for execution by the one or more processors:
collecting or obtaining one or more organic molecule signatures of a biological sample of a plurality of training subjects, wherein a first subset of training subjects in the plurality of training subjects have a first phenotype associated with the one or more organic molecule signatures, and a second subset of training subjects in the plurality of training subjects have a second phenotype associated with the one or more organic molecule signatures; and
training the untrained or partially untrained machine learning algorithm or predictive model with the one or more organic molecule signatures of the plurality of training subjects and the corresponding first phenotype and second phenotype associated with the one or more organic molecular signatures, thereby producing or generating a trained predictive model.
